(19) United States
(12) Patent Application Publication
(10) Pub. No.: US 2024/0218328 A1
(43) Pub. Date: Jul. 4, 2024

(54) COMPOSITIONS AND METHODS FOR DIFFERENTIATING AND EXPANDING B LINEAGE CELLS

(71) Applicant: STEMCELL TECHNOLOGIES CANADA INC., Vancouver (CA)

(72) Inventors: Nooshin TABATABAEI-ZAVAREH, Vancouver (CA); Patrick BRAUER, Vancouver (CA)

(21) Appl. No.: 18/557,367

(22) PCT Filed: Apr. 29, 2022
(86) PCT No.: PCT/CA2022/050662

§ 371 Date: Oct. 26, 2023

Related U.S. Application Data
(60) Provisional application No. 63/182,054, filed on Apr. 30, 2021.

(51) Int. Cl. C12N 5/0781

(52) U.S. Cl. CPC C12N 5/0635; C12N 2500/90; C12N 2501/125; C12N 2501/145; C12N 2501/2302; C12N 2501/2303; C12N 2501/2304; C12N 2501/2306; C12N 2501/2307; C12N 2501/231; C12N 2501/2321; C12N 2501/26; C12N 2501/52; C12N 2506/11; C12N 2506/45

(57) ABSTRACT

Disclosed are media, kits and methods for the directed differentiation of cells to the B cell lineage. The disclosed differentiation approaches may take primary cells or pluripotent stem cell-derived cells through one or more intermediate cell populations to yield the B lineage cells, using one or more stage-specific media formulations. Thus, media and supplements for carrying out directed differentiation workflows may be comprised in a kit that contains one or more basal media and one or more supplements to be added thereto.

### RELATED APPLICATION

This application claims the benefit of priority of U.S. Provisional Patent Application No. 63/182,054, filed Apr. 30, 2021, the entire content of which is hereby incorporated by reference in its entirety.

### TECHNICAL FIELD

This disclosure relates to cell culture applications, and more specifically to cell culture applications using hematopoietic cells, and still more specifically to cell culture applications related to one or more population(s) of B cells.

### BACKGROUND

The blood of mammals is composed of various cell types, including lymphocytes, thrombocytes, erythrocytes, and the direct and indirect precursors thereof. Leukocytes may be referred to as white blood cells, and these function in the immune system of the host. Leukocytes can be further subdivided into B cells, T cells, NK cells, monocytes, macrophages, dendritic cells, eosinophils, basophils, and neutrophils. Each of such leukocytes perform specific functions in the immune system of the host.

B cells, as with other blood cells, derive from a hematopoietic stem/progenitor cell (HSPC) that is capable of self-renewal and differentiating to each blood cell lineage. B cells are central components of humoral immunity and secrete antibodies upon binding an antigen (via B cell receptors expressed on the surface thereof).

In vivo, mammalian B cells develop in the bone marrow, and beginning from an HSPC progress through various stages of development, including a pro-B cell, pre-B cell, and an immature B cell. Immature B cells mature into memory B cells in a second lymphoid organ (e.g. spleen, thymus, etc.), and into plasmablasts and plasma cells (i.e. antibody-producing cells) in either a second lymphoid organ or the bone marrow. Recapitulating these developmental steps in vitro or ex vivo has achieved only limited success. In particular, it has not been possible to recapitulate these developmental steps in a serum-free and feeder-cell free culture system using either tissue-derived cells or pluripotent stem cells (“PSC”) as the originating material.

Both primary tissue-derived cells and PSCs, in particular, provide an opportunity to create homogenous, customizable, large-scale populations of B lineage cells appropriate for clinical applications. Differentiated PSC also enable gene-engineering methods that facilitate disease modeling or cell therapy applications.

Given their involvement in sensing antigens in their environment and, upon stimulation, to secrete large quantities of antibodies to neutralize the target, B cells are the subject of intense research and therapeutic interest. Accordingly, there is a need for efficient means of obtaining immature and mature B lineage cells in culture from precursor populations, whether originating from PSCs or from appropriate precursors isolated from cord blood or bone marrow.

### SUMMARY

This disclosure relates to media compositions and/or supplements to be added into a medium, and to methods for culturing/differentiating hematopoietic stem/progenitor cells (HSPC). More specifically, this disclosure relates to methods of step-wisedly differentiating HSPC into various B cell lineages using stage-specific media and/or supplements to be added to a basal medium.

In one aspect of this disclosure are provided directed differentiation methods for preparing a population of B cell precursors, comprising contacting a population of CD34⁺ hematopoietic stem or progenitor cells (HSPC) with a derivation medium comprising a basal medium, at least one of stem cell factor (SCF), thrombopoietin (TPO), and FMS-like tyrosine kinase 3 ligand (FLT3L), and at least one other cytokine; and culturing the population of HSPC in the derivation medium under serum-free conditions to obtain a population of B cell precursors.

In one embodiment, the at least one other cytokine is one or more of IL-3, IL-6, or IL-7. In one embodiment, the at least one other cytokine is one or more of IL-2, IL-3, IL-4, IL-6, IL-7, IL-10, or IL-21.

In one embodiment, the population of HSPC are enriched from cord blood or bone marrow, or are differentiated from pluripotent stem cells (PSC).

In one embodiment, the population of B cell precursors express one or both of CD10 or CD19.

In one embodiment, the derivation medium comprises either SCF or TPO.

In one embodiment, the methods may further comprise contacting the population of B cell precursors with a differentiation medium and culturing the population of B cell precursors in the differentiation medium under serum-free conditions. In one embodiment, the method may further comprise obtaining a population of CD19⁺ B lineage cells.

In one embodiment, the methods may further comprise obtaining more CD19⁺ B lineage cells than after culturing the population of HSPC in the derivation medium.

In one embodiment, at least a fraction of the CD19⁺ B lineage cells are IgM⁺ cells.

In one embodiment, the differentiation medium comprises a basal medium, at least one of SCF, TPO, and FLT3L, and the at least one other cytokine.

In one embodiment, the methods may further comprise contacting the population of CD19⁺ B lineage cells with a downstream differentiation medium and culturing the population of CD19⁺ B lineage cells in the downstream differentiation medium under serum-free conditions. In one embodiment, the methods may further comprise obtaining more IgM⁺ cells than after culturing the population of B cell precursors in the differentiation medium.

In one embodiment, at least a fraction of the IgM⁺ cells are antibody secreting cells.

In one embodiment, the downstream differentiation medium comprises a basal medium, a ligand of human CD40, and the at least one other cytokine.

In one embodiment, the methods are performed under feeder cell-free conditions. In one embodiment, feeder cell-free conditions comprise an extracellular matrix protein or a cell adhesion molecule. In one embodiment, feeder-cell free conditions are in the absence of an extracellular matrix protein or a cell adhesion molecule that is solubilized or coated on a surface of a culture vessel.

In one embodiment, the extracellular matrix protein or the cell adhesion molecule is solubilized or coated on a surface of a culture vessel. In one embodiment, the extracellular matrix protein or the cell adhesion molecule is a fibronectin, a vitronectin, a laminin, an ECM1, a SPARC, an osteopontin, a vascular cell adhesion molecule, an immobilized SCF protein, or any combination of the foregoing.

In another aspect of this disclosure are provided directed differentiation methods for preparing a population of B lineage cells comprising contacting a population of B cell precursors with a differentiation medium comprising a basal medium, at least one of SCF, TPO and FLT3L, and at least one other cytokine, and culturing the population of B cell precursors in the differentiation medium under serum-free conditions to obtain a population of B lineage cells.

In one embodiment, the at least one other cytokine is one or more of IL-3, IL-6, or IL-7. In one embodiment, the at least one other cytokine is one or more of IL-2, IL-3, IL-4, IL-6, IL-7, IL-10, or IL-21.

In one embodiment, the population of B cell precursors express one or both of CD10 or CD19.

In one embodiment, the population of B cell precursors are derived from a population of CD34⁺ hematopoietic stem or progenitor cells (HSPC) that are either enriched from cord blood or bone marrow, or are differentiated from pluripotent stem cells (PSC).

In one embodiment, the population of B lineage cells express CD19. In one embodiment, the population of B lineage cells comprises more CD19⁺ cells than after culturing the population of HSPC in a derivation medium to yield the population of B cell precursors.

In one embodiment, the derivation medium is serum-free. In one embodiment, the derivation medium comprises a basal medium, at least one cytokine, and one or more of SCF, TPO, and FLT3L.

In one embodiment, at least a fraction of CD19⁺ B lineage cells are IgM⁺ cells.

In one embodiment, the methods may further comprise contacting the population of B lineage cells with a downstream differentiation medium and culturing the population of B lineage cells in a downstream differentiation medium under serum-free conditions. In one embodiment, the methods may further comprise obtaining more IgM⁺ cells than after culturing the population of B cell precursors in the differentiation medium.

In one embodiment, at least a fraction of the IgM⁺ cells are antibody secreting cells.

In one embodiment, the downstream differentiation medium comprises a basal medium, a ligand of human CD40, and the at least one other cytokine.

In one embodiment, the methods are performed under feeder cell-free conditions. In one embodiment, feeder cell-free conditions comprise an extracellular matrix protein or a cell adhesion molecule. In one embodiment, feeder-cell free conditions are in the absence of an extracellular matrix protein or a cell adhesion molecule that is solubilized or coated on a surface of a culture vessel.

In one embodiment, the extracellular matrix protein or the cell adhesion molecule is solubilized or coated on a surface of a culture vessel. In one embodiment, the extracellular matrix protein or the cell adhesion molecule is a fibronectin, a vitronectin, a laminin, an ECM1, a SPARC, an osteopontin, a vascular cell adhesion molecule, an immobilized SCF protein, or any combination of the foregoing.

In another aspect of this disclosure are provided kits for the directed differentiation of B lineage cells, the kit comprising a basal medium, and at least one supplement. In one embodiment, the at least one supplement comprises at least one of stem cell factor (SCF), thrombopoietin (TPO), and FMS-like tyrosine kinase 3 ligand (FLT3L), and at least one other cytokine.

In one embodiment, the kits further comprise a second supplement.

In one embodiment, the second supplement comprises at least one of stem cell factor (SCF), thrombopoietin (TPO), and FMS-like tyrosine kinase 3 ligand (FLT3L), and at least one other cytokine.

In one embodiment, a formulation of the at least one supplement is different from the second supplement.

In one embodiment, the kit further comprises a third supplement. In one embodiment, the third supplement comprises a ligand of human CD40 and at least one other cytokine.

In one embodiment, the at least on cytokine is one or more of IL-2, IL-3, IL-4, IL-6, IL-7, IL-10, or IL-21.

In other aspects of this disclosure are provided media for the directed differentiation of B cell precursors, or B lineage cells, or cells downstream of B lineage cells, such as IgM⁺ cells and/or antibody secreting cells.

In one aspect of this disclosure are provided media for the derivation of B cell precursors. In one embodiment, a derivation medium comprises a basal medium at least one of stem cell factor (SCF), thrombopoietin (TPO), and FMS-like tyrosine kinase 3 ligand (FLT3L), and at least one other cytokine.

In one aspect of this disclosure are provided media for the differentiation of B lineage cells. In one embodiment, a differentiation medium comprises a basal medium at least one of stem cell factor (SCF), thrombopoietin (TPO), and FMS-like tyrosine kinase 3 ligand (FLT3L), and at least one other cytokine.

In one aspect of this disclosure are provided media for the downstream differentiation of cells downstream of B lineage cells. In one embodiment, a downstream differentiation medium comprises a basal medium, a ligand of human CD40 and at least one other cytokine.

In one embodiment, the at least on cytokine included in media of this disclosure is one or more of IL-2, IL-3, IL-4, IL-6, IL-7, IL-10, or IL-21.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows representative flow cytometry plots of CD34⁺ HSPC enriched from a single human cord blood donor sample. An enriched population of CD34⁺ cells (ie “bulk”) were stained and then sorted for Lin⁻ (Lin included CD3, CD14, CD15, CD16, CD19, CD56, and CD66b) (A). The cells of (A) were further sorted to obtain two more specialized populations of putative progenitors of B cell fate: Lin⁻CD34⁺CD38^{-/lo/mid}CD10⁻ cells (“Population 1” or “pop1”) in (B); and Lin⁻CD34⁺CD38^{mid}CD10⁺ cells (“Population 2” or “pop2”) in (B). Pop1 cells have multiple potential for granulocytes, monocytes, lymphoid and erythroid progeny, while pop2 cells are restricted to lymphoid lineage.

FIG. 2 shows bar graphs summarizing the results of B cell precursor derivation from a population of CD34⁺ HSPC. Pop2 cells were cultured for 14 days in different derivation medium formulations (“SUPPLCTL” baseline medium, and formulations lacking the indicated cytokine/growth factor) testing the effect of the absence of selected growth factors or cytokines. After 14 days, the frequency and yield of B cell precursors (A) of cells expressing CD19⁺ (B) was determined. The results shown are mean of 1-3 independent experiment.

FIG. 3 shows bar graphs summarizing the results of B cell precursor derivation from populations of CD34⁺ HSPC. Pop1 and pop2 cells were separately cultured for 14 days in derivation medium excluding TPO but further comprising indicated cytokines, either alone or in combination. For pop2-derived cells, the bar graphs show: overall fold expansion (A), the frequency and yield of B cell precursors (B), the frequency and yield of cells expressing CD19⁺ (C), and a sample flow plot analyzing CD10 and CD19 expression (D). For pop1 cells, the bar graphs show: overall fold expansion (E), the frequency and yield of B cell precursors (F), the frequency and yield of CD19⁺ B lineage cells (G), and a sample flow plot analyzing CD10 and CD19 expression (H). The results shown are mean of 2-8 independent experiments.

FIG. 4 shows bar graphs summarizing the results of CD19⁺ B lineage cell differentiation from a population of CD34⁺ HSPC-derived B cell precursors. Pop2 cells were first cultured for 14 days in the control formulation of FIG. 2, and then transitioned to various differentiation medium formulations (“DiM”) that excluded individual and combinations of factors, as indicated, for an additional 14 days. After 28-days in culture, the frequency and yield of B cell precursors (A), CD19⁺ B lineage cells (B), and IgM⁺ cells (C) among the output cells was determined. The results shown are mean of 1-3 independent experiment.

FIG. 5 shows bar graphs comparing the differentiation efficiencies of CD34⁺ HSPCs in media of this disclosure. Pop1 cells and pop2 cells were separately cultured for 14 days in two versions of derivation media, and then transitioned to a differentiation medium formulation of FIG. 4 for 14 more days. For pop2-derived cells, the bar graphs show: the frequency and yield of B cell precursors (A), the frequency and yield of CD19⁺ B lineage cells (B), and the frequency and yield of IgM⁺ cells as a function of the CD19⁺ fraction of cells (C). For pop 1 cells, the bar graphs show: the frequency and yield of B cell precursors (D), the frequency and yield of CD19⁺ B lineage cells (E), and the frequency and yield of IgM⁺ cells as a function of the CD19⁺ fraction of cells (F). The results shown are mean of 4 independent experiments.

FIG. 6 shows bar graphs summarizing the results of CD19⁺ B lineage cell differentiation from a population of CD34⁺ HSPC-derived B cell precursors. Pop1 cells were first cultured for 14 days in a derivation medium formulation shown in FIG. 2, but different from those used in FIGS. 3-5. The resulting B-cell precursors were transitioned to a differentiation medium of this disclosure for 14 more days. After 28 days, the frequency and yield of B cell precursors (A) and CD19⁺ B lineage cells (B) was determined. The results shown are mean of 1-5 independent experiments+/−the standard error.

FIG. 7 shows bar graphs comparing the effect of extended culture on the differentiation of B lineage cells. Pop1 cells were first cultured for 14 days in derivation medium, and then transitioned to differentiation medium for either 14 or 28 more days. The bar graphs shows the frequency and yield of CD19⁺ B lineage cells (A) and of IgM⁺ cells as a function of the CD19⁺ fraction of cells (B). The results shown are mean of 4 independent experiments+/−the standard error.

FIG. 8 shows a bar graph summarizing the frequency and yield of day-28 CD19⁺ cells, differentiated using media formulations and methods of this disclosure, starting from either bulk CD34⁺ cells, pop1 CD34⁺ cells, or pop2 CD34⁺ cells. Bars represent the mean of at least 23 independent data points.

FIG. 9 shows the results of experiments to optimize the differentiation efficiency of IgM⁺ cells from CD19⁺ B lineage cells. Pop1 or bulk cells were separately cultured for 14 days in a derivation medium and transferred to a differentiation medium for a further 14 days. Thereafter, the day 28 cells were separately cultured in various downstream differentiation medium (“DDM”) formulations: a negative control and two formulations including a ligand of human CD40 and different cytokines/growth factors. Day 35 pop1-derived cells were analyzed for frequency and yield of CD19⁺ B lineage cells (A) and IgM⁺ cells as a function of the CD19⁺ fraction of cells (B). The results shown are mean of at least 4 independent experiments. Day 35 pop1-derived cells were also tested for IgM (red/grey) and IgG (blue/black) secretion in an ELISPOT assay. Representative images along with the number of output antibody secreting cells are shown for control cells (Ci) and cells cultured in the indicated downstream differentiation media (Cii and Ciii). Day 35 bulk-derived cells were analyzed for frequency and yield of CD19⁺ B lineage cells (D) and IgM⁺ cells as a function of the CD19⁺ fraction of cells (E). The results shown are mean of at least 3 independent experiments.

FIG. 10 shows the results of deriving B cell precursors from a population of human PSC-derived CD34⁺ HSPC. PSC-derived CD34⁺ HSPC were cultured for 14 days in a derivation medium of this disclosure either in the absence or presence of different coating materials, as indicated. The results shown are mean of at least 2 independent experiments (A). The day 28 population of cells cultured in the absence or presence of different coating materials was analyzed by flow cytometry for CD10 and CD19 expression (B). PSC-derived or cord blood-derived bulk cells were analyzed by qRT-PCR for the expression of EBF1 (C) and PAX5 (D) at the indicated time points of the derivation/differentiation protocol described herein. The day 28 population of cells was analyzed by flow cytometry for CD10 and CD19 expression and CD19 and CD20 expression (E). Bars represent the mean of at least 1 experiment+/−the standard error.

FIG. 11 shows bar graphs summarizing the effects of different coatings on the derivation/differentiation of cord blood-derived CD34⁺ cells. Pop1 cells were cultured for 14 days in a derivation medium on the indicated coatings and the frequency and yield of B cell precursors (A) and of cells expressing CD19 (B) was determined. Day 14 cells were cultured for an additional 14 days in a differentiation medium on the indicated coatings, and the frequency and yield of day 28 CD19⁺ B lineage cells (C) and the frequency of IgM⁺ cells as a function of the CD19⁺ fraction of cells (D) was determined. The results shown are mean of at least 2 independent experiments.

### DETAILED DESCRIPTION

This disclosure relates to media compositions and/or supplements to be added into a medium, and to methods for culturing/differentiating HSPC. More specifically, this disclosure relates to differentiating HSPC into various B cell lineages using stage-specific media and/or supplements to be added to a basal medium.

Where used herein the term “hematopoietic stem or progenitor cell” or “HSPC” refers to a cell of the hematopoietic lineage that is capable of self-renewal and/or differentiating into a more specialized cell of the hematopoietic lineage. In some embodiments, HSPC may be comprised in a population of cells, which population of HSPC may be >50% pure, >60% pure, >70% pure, >80% pure, or >90% pure. HSPC may be obtained from bone marrow (BM), umbilical cord blood (CB), embryonic through to adult peripheral blood (PB), thymus, peripheral lymph nodes, gastrointestinal tract, tonsils, gravid uterus, liver, spleen, placenta, or any other tissue having localized populations of HSPC. In some embodiments, HSPC (or, the population of HSPC) are enriched from a tissue source or another population of cells comprising HSPC, such as by immunomagnetic separation or fluorescence activated cell sorting. HSPC may also be differentiated from pluripotent stem cells, such as induced pluripotent stem cells, embryonic stem cells, naïve stem cells, extended stem cells, or the like. A hallmark of HSPC is the expression of the transmembrane phosphoglycoprotein CD34, thus HSPC may be referred to as CD34⁺ cells. Human HSPCs are further defined by expression of CD45 and CD34, and may be still further defined by combinations of markers such as CD38, CD43, CD45RO, CD45RA, CD10, CD49f, CD59, CD90, CD109, CD117, CD133, CD166, HLA-DR, CD201, and integrin-alpha3 which may be used to distinguish HSPC subsets. HSPCs may lack expression, or have only low expression, of markers such as Glycophorin A, CD3, CD4, CD8, CD14, CD15, CD19, CD20 and CD56; such markers may characterize more mature blood cells.

Where used herein the term “pluripotent stem cell” or “PSC” refers to a cell that is capable of self-renewal and/or differentiating to any cell type of any of the three embryonic germ layers. PSC, such as embryonic stem cells, may be isolated from a blastocyst and subjected to either maintenance or differentiation cell culture conditions. PSC, such as induced pluripotent stem cells, may be derived from any cell type by the forced expression of certain pluripotency genes, such as Oct4, Nanog, Sox2, Klf4, etc. Expression of pluripotency genes may be forced by introducing their coding regions, whether stably or transiently, into a host cell or by introducing factors that activate endogenous copies of such genes.

Where used herein, the term “B cell precursor” or “B cell precursors” refers to a cell type that is more specialized than a HSPC, but is capable of further differentiating into one or more lymphoid cell types, such as B cells. A B cell precursor may be a direct descendant of a HSPC, whether tissue- or PSC-derived, or may be further removed from a HSPC. Further, a B cell precursor may directly differentiate into a downstream lymphoid cell type, such as a B cell, or may undergo one or more further steps of differentiation before becoming a B cell. One example of a B cell precursor is a cell that is positive for one of the phenotypic markers CD10 or CD19. In one example, a CD10⁺ B cell precursor is negative for CD19, and such a cell may not be committed to the B lineage. In one example, a CD19⁺ B cell precursor is negative for CD10, and such a cell may be committed to the B lineage. Other phenotypic markers that may be expressed by B cell precursors include CD20, CD45RA, CD34, CD38, CD161, CD122, CD117, CD127, and/or integrinβ7. Further, examples of phenotypic markers that may not be expressed by B cell precursors include CD10, CD19, CD20, CD45RA, CD34, CD38, CD161, CD122, CD117, CD127, and/or integrinβ7. Herein, unless explicitly stated, a population of B cell precursors may refer to a homogeneous population of cells or a heterogeneous population of cells capable of differentiating to one or more downstream cell types. In one embodiment, a B cell precursor may be capable of differentiating into any type of B lineage cell. In one embodiment, a B cell precursor may be more restricted in its differentiation capacity, such as to only differentiate into double positive CD10⁺CD19⁺ B lineage cells.

Where used herein, the term “B lineage cell” refers to a type of lymphocyte of the hematopoietic lineage that may be differentiated from HSPC, whether tissue- or PSC-derived, and is more specialized/committed than a B cell precursor. More specifically, B lineage cells may derive from multilymphoid progenitors (MLPs) or common lymphoid progenitors (CLPs). Earlier B lineage cells may be characterized by expression of both CD10 and CD19 surface markers, and more mature naïve B cells express CD19 but not CD10. In some embodiments, B lineage cells may express CD19 (and not CD10), but may nevertheless be distinguishable from B cell precursors on the basis of one or more other marker. In one embodiment, double positive CD10⁺+CD19⁺ B lineage cells may lose expression of one or the other marker, but may nevertheless remain committed to the B lineage. In one embodiment, after deriving B cell precursors in accordance with this disclosure some CD19⁺ cells may be included thereamong, and such CD19⁺ cells may be B lineage cells or may be a more primitive subset of cells; nevertheless, the frequency of B lineage cells will increase upon exposing to a differentiation medium those cells obtained after exposure to derivation media. Said another way, a population of B lineage cells may be characterized by a higher frequency of cells expressing CD19 compared to a population of B cell precursors.

Where used herein, the term “B cell” or “B cells” refers to a cell type that is differentiated from a B lineage cell. B cells are typically characterized by: the absence of T-, NK-, and erythromyeloid-specific markers; the expression of one or more of CD19, CD20, B cell receptor (i.e. surface IgM), IgG, IgD, IgA, IgE; CD138; and their effector functions. More specifically, effector functions of B cells may include the production of antibodies. Plasma cells and plasmablasts, types of B cells, secrete antibodies and are characterized by the expression of CD138 (plasma cells), CD38 and CD27. The differentiation of B cells from PSC or HSPC is usually intermediated by one or more progenitor populations, such as a PSC-derived mesodermal precursor and/or lymphoid progenitor cells (e.g. PSC-derived lymphoid progenitors).

### Media and Methods

The methods of this disclosure encompass those steps for differentiating either tissue- or PSC-derived hematopoietic progenitors through to immature or mature B cells via one or more intermediate population of cells. The methods disclosed herein for differentiating HSPC, and other derived downstream cell types, are preferably in vitro methods. Media of this disclosure, as disclosed below, may be used to perform the methods of this disclosure, and are thus independent aspects of this disclosure.

A directed differentiation method of this disclosure may comprise contacting a population of CD34⁺ HSPC with a derivation medium, and culturing the population of HSPC in the derivation medium for a time sufficient to derive the population of B cell precursors. In some embodiments, limited differentiation to B lineage cells may also occur during this stage.

In one embodiment, directed differentiation methods of this disclosure derive a population of B cell precursors. In one embodiment, directed differentiation methods of this disclosure derive a population of B lineage cells. In one embodiment, directed differentiation methods of this disclosure derive a population of IgM⁺ and/or antibody secreting cells.

A derivation medium is any medium that may be used to differentiate HSPC to a population of B cell precursors. In a preferred embodiment, the derivation medium is serum-free. If derivation media are serum-free, it may be necessary to include in such media a serum replacement supplement, such as BIT 9500 Serum Substitute (STEMCELL Technologies, Catalogue #09500), or other commercially available serum replacement solutions. Alternatively, components ordinarily present in serum that are needed for culturing or differentiating any cells of this disclosure may be individually added at acceptable concentrations into derivation media. In one embodiment, a component ordinarily present in serum is albumin. If an albumin is included in the derivation medium (in place of serum), it may be from any species, but is typically either bovine or human. In some embodiments, an albumin may be recombinant.

Derivation media of this disclosure, alternatively referred to as “SUPPLCTL” media, will include a basal medium that is formulated as appropriate to culture the HSPC and to support derivation of B-cell precursors. Thus, a suitable basal medium is any basal medium that is supportive of culturing cells of the hematopoietic lineage, and in particular B cell precursors and/or B lineage cells and/or IgM⁺ cells. Exemplary basal media include, but are not limited to, STEMdiff™ Hematopoietic-EB Basal Medium (STEMCELL Technologies, Catalogue #100-171), STEMdiff™ Hematopoietic Basal Medium (STEMCELL Technologies, Catalogue #05311), STEMdiff™ APEL™2 Medium (STEMCELL Technologies, Catalogue #05270), StemSpan™ AOF Medium (STEMCELL Technologies, Catalogue #100-0130), StemSpan™ SFEM & SFEM II, ImmunoCult™ XF (STEMCELL Technologies, Catalogue #09650, 09655, 10981), or any other commercially available basal medium fit for the purpose. Common components formulating basal media may include salts, buffers, lipids, amino acids, trace elements, certain proteins, vitamins, minerals, reducing agents, etc. In one embodiment, basal media are optimized to support the differentiation of HSPC and the derivation of B cell precursor(s) therefrom.

In one embodiment, derivation media comprise at least one of stem cell factor (SCF), thrombopoietin (TPO), and FMS-like tyrosine kinase 3 ligand (FLT3L). In one embodiment, derivation medium includes two or more of SCF, TPO, and FLT3L. In one embodiment, derivation medium includes each of SCF, TPO, and FLT3L. In one embodiment, derivation media comprise either SCF or TPO. In one embodiment, derivation media do not include one, two, or each of TPO, SCF, and FLT3L. In one embodiment, derivation media do not include one or both TPO and SCF. In one embodiment, derivation media do not include TPO. In one embodiment, derivation media do not include SCF.

If SCF is included in a derivation medium, a concentration of SCF therein may range between about 0.5 ng/mL and 500 ng/mL, between about 1 ng/mL and 250 ng/mL, between about 5 ng/mL and 100 ng/mL, or between about 10 ng/mL and 50 ng/mL.

If FLT3L is included in a derivation medium, a concentration of FLT3L therein may range between about 0.5 ng/mL and 500 ng/mL, between about 1 ng/mL and 250 ng/mL, between about 5 ng/mL and 100 ng/mL, or between about 10 ng/mL and 50 ng/mL.

If TPO is included in a derivation medium, a concentration of TPO therein may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, derivation media further comprise at least one other cytokine. In one embodiment, the at least one other cytokine is one or more interleukin. In one embodiment, the at least one other cytokine is one or more of IL-1, IL-2, IL-3, IL-4, IL-6, IL-7, IL-10, IL-15, IL-17, and IL-21. In one embodiment, the at least one other cytokine is IL-3, IL-6, or IL-7, or any combination thereof.

A concentration of the at least one other cytokine comprised in a derivation medium may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, derivation media further comprise one or more additional cytokines or growth factors, or small molecules, to further enhance the derivation of a population of B cell precursors from a population of HSPC. Non-limiting examples of the one or more additional cytokines or growth factors that may be included in a derivation medium include erythropoietin (EPO), insulin growth factor 1 (IGF-1) and insulin growth factor 2 (IGF-2), B cell activating factor (BAFF), a proliferation-inducing ligand (APRIL), and interferon gamma (IFN-g).

A concentration of the one or more additional growth factors comprised in a derivation medium may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, derivation media may be formulated as a complete medium. In one embodiment, derivation media may be prepared freshly before use, and thus the basal medium may be stored separately from one or more supplements to be added to the basal medium. In one example, the growth factors and cytokines may be combined in one or more supplements to be added to the basal medium just prior to use of the complete derivation medium in a derivation/differentiation method.

The growth factors and cytokines to be included in a derivation medium may be sourced from various commercial suppliers, and may be recombinant.

Derivation media of this disclosure may synergize with a substrate for supporting the culture of the population of HSPC. In one embodiment, stromal or feeder cells may be used together with cell culture media of this disclosure. Non-exhaustive examples of such cells include the embryonic liver cell line EL08.1D2, AFT024 cells, OP9 cells, MS-5 or M2-10B4 cells, mouse embryonic fibroblasts or stromal cells from embryonic aorta-gonad mesonephros (AGM).

In one embodiment, culturing a population of HSPC is done under feeder cell-free and/or stromal cell-free conditions. Such approaches may utilize medium previously conditioned by stromal/feeder cells, or such a system may utilize a stroma/feeder cell replacement. A stroma/feeder cell replacement may comprise one or more defined components that provide appropriate signals or contact sites to cells in culture. Such components may be included (e.g. solubilized) in a derivation medium or employed as a coating applied to an inner culture surface of a culture vessel or on solid surfaces suspended in cell culture media, such as on particles, beads, microcarriers, or the like. Non-exhaustive examples of such components may include fibronectin coatings, gelatin coatings, collagen coatings, an immobilized Notch ligand, or coatings such as StemSpan™ Lymphoid Differentiation Coating Supplement (STEMCELL Technologies, Catalogue #09925) or Matrigel (Corning).

In one embodiment, culturing the population of HSPC is in the presence of an extracellular matrix protein or a cell adhesion molecule (while in the absence of feeder cell and/or stroma cell support). In one embodiment, the extracellular matrix protein or the cell adhesion molecule is solubilized in a derivation medium or coated on a surface in contact with the derivation medium. In one embodiment, the extracellular matrix protein is a fibronectin, a vitronectin, a laminin, ECM1, SPARC, or osteopontin. In one embodiment, the cell adhesion molecule is a vascular cell adhesion molecule (e.g. VCAM-1) or an immobilized SCF protein (e.g. SCF-Fc).

In one embodiment, different combinations of the foregoing proteins may be used. In one embodiment, combinations of extracellular matrix proteins are used. In one embodiment, combinations of cell adhesion molecules are used. In one embodiment, combinations of extracellular matrix protein(s) and cell adhesion molecule(s) are used.

Where used, a concentration of an extracellular matrix protein or a cell adhesion molecule that synergizes with a derivation medium ranges between about 0.1 to 100 μg/mL (or 0.03 to 30 μg/well of a 96-well plate), between about 0.2 to 50 μg/mL (or 0.06 to 15 μg/well of a 96-well plate), or between about 0.5 to 20 μg/mL (or 0.15 to 6 μg/well of a 96-well plate).

In one embodiment, media of this disclosure are not dependent upon use together with an extracellular matrix protein or a cell adhesion molecule.

Culturing the population of HSPC in a derivation medium may be for any period of time that does not impact their viability or capacity to differentiate to downstream lineages. In one embodiment, a directed differentiation method for deriving a population of B cell precursors from a population of HSPC comprises culturing a population of CD34⁺ HSPC in derivation medium for between about 1 and 28 days, between about 3 and 25 days, between about 5 and 21 days, or between about 7 and 14 days. In one embodiment, a directed differentiation method for deriving a population of B cell precursors from a population of HSPC comprises culturing a population of CD34⁺ HSPC in derivation medium for between about 3 and 14 days.

In one embodiment, 1% or more of the derived cells are B cell precursors (e.g. express CD10). In one embodiment, 5% or more of the derived cells are B cell precursors. In one embodiment, 10% or more of the derived cells are B cell precursors. In one embodiment, 20% or more of the derived cells are B cell precursors. In one embodiment, 30% or more of the derived cells are B cell precursors. In one embodiment, 40% or more of the derived cells are B cell precursors. In one embodiment, 50% or more of the derived cells are B cell precursors. Further, in one embodiment, 1% or more of cells derived using a derivation medium express CD19. In one embodiment, 5% or more of cells derived using a derivation medium express CD19. In one embodiment, 10% or more of cells derived using a derivation medium express CD19. In one embodiment, 20% or more of cells derived using a derivation medium express CD19. In one embodiment, 30% or more of cells derived using a derivation medium express CD19. In one embodiment, 40% or more of cells derived using a derivation medium express CD19.

In one embodiment, the CD19⁺ cells that may appear on derivation of B cell precursors (using derivation media) may be B lineage cells. In one embodiment, the CD19⁺ cells that may appear on derivation of B cell precursors (using derivation media) may not be B lineage cells, but rather a more primitive cell or a progenitor thereof. In one embodiment, both of the foregoing types of cells may be comprised in the cells obtained after culture in a derivation medium.

In one embodiment, derivation of B cell precursors using a derivation medium yields 1 or more CD10⁺ cells per input cell, 5 or more CD10⁺ cells per input cell, 10 or more CD10⁺ cells per input cell, 20 or more CD10⁺ cells per input cell, 50 or more CD10⁺ cells per input cell, or 100 or more CD10⁺ cells per input cell. Further, in one embodiment, derivation of B cell precursors using a derivation medium yields 1 or more CD19⁺ cells per input cell, 5 or more CD19⁺ cells per input cell, 10 or more CD19⁺ cells per input cell, 20 or more CD19⁺ cells per input cell, 50 or more CD19⁺ cells per input cell, or 100 or more CD19⁺ cells per input cell.

In another aspect, the directed differentiation methods of this disclosure further comprise contacting a population of B cell precursors with a differentiation medium, and culturing the population of B cell precursors in the differentiation medium for a time sufficient to obtain B lineage cells.

In one embodiment, the population of B cell precursors express one or both of CD10 or CD19.

In one embodiment, the B lineage cells are CD19⁺ cells. In one embodiment, the B lineage cells are double positive CD10⁺CD19⁺. In one embodiment, the population of B lineage cells comprises more CD19⁺ cells than after culturing the population of CD34⁺ HSPC in a derivation medium (i.e. following derivation of a population of B cell precursors using derivation media). In one embodiment, the population of B lineage cells comprises 2-fold or more CD19⁺ cells than following derivation of a population of B cell precursors. In one embodiment, the population of B lineage cells comprises 3-fold or more CD19⁺ cells than following derivation of a population of B cell precursors. In one embodiment, the population of B lineage cells comprises 4-fold or more CD19⁺ cells than following derivation of a population of B cell precursors. In one embodiment, the population of B lineage cells comprises 5-fold or more CD19⁺ cells than following derivation of a population of B cell precursors. In one embodiment, the population of B lineage cells comprises 10-fold or more CD19⁺ cells than following derivation of a population of B cell precursors. In one embodiment, the population of B lineage cells comprises 20-fold or more CD19⁺ cells than following derivation of a population of B cell precursors. In one embodiment, the population of B lineage cells comprises 50-fold or more CD19⁺ cells than following derivation of a population of B cell precursors.

A differentiation medium is any medium that may be used to differentiate a population of B cell precursors (to a population of B lineage cells). In a preferred embodiment, the differentiation medium is serum-free. If differentiation media are serum-free, it may be necessary to include in such media a serum replacement supplement, such as BIT 9500 Serum Substitute (STEMCELL Technologies, Catalogue #09500), or other commercially available serum replacement solutions. Alternatively, components ordinarily present in serum that are needed for culturing or differentiating any cells of this disclosure may be individually added at acceptable concentrations into a differentiation medium. In one embodiment, a component ordinarily present in serum is albumin. If an albumin is included in a differentiation media (in place of serum), it may be from any species, but is typically either bovine or human. In some embodiments, an albumin may be recombinant.

Differentiation media of this disclosure, alternatively referred to as “DiM” media, will include a basal medium formulated as appropriate to culture HSPC and/or B-cell precursors and/or B-lineage cells and/or IgM⁺ cells. Thus, a suitable basal medium is any basal medium that is supportive of culturing cells of the hematopoietic lineage, and in particular B lineage cells. Exemplary basal media include, but are not limited to, STEMdiff™ Hematopoietic-EB Basal Medium (STEMCELL Technologies, Catalogue #100-171), STEMdiff™ Hematopoietic Basal Medium (STEMCELL Technologies, Catalogue #05311), STEMdiff™ APEL™2 Medium (STEMCELL Technologies, Catalogue #05270), StemSpan™ AOF Medium (STEMCELL Technologies, Catalogue #100-0130), StemSpan™ SFEM & SFEM II, ImmunoCult™ XF (STEMCELL Technologies, Catalogue #09650, 09655, 10981), or any other commercially available basal medium fit for the purpose. Common components used to formulate such basal media may include salts, buffers, lipids, amino acids, trace elements, certain proteins, vitamins, minerals, reducing agents, etc. In one embodiment, basal media are optimized to support the differentiation of HSPC and the derivation of B-cell precursor(s) therefrom, and the further differentiation of B lineage cells.

In one embodiment, differentiation media comprise at least one of FLT3L, TPO, and SCF. In one embodiment, differentiation media include two or more of FLT3L, TPO, and SCF. In one embodiment, differentiation media include each of FLT3L, TPO, and SCF. In one embodiment, differentiation media include each of FLT3L, TPO, and SCF, and at least one other cytokine. In one embodiment, differentiation media comprise either SCF or TPO. In one embodiment, differentiation media do not include one or each of TPO, SCF, and FLT3L. In one embodiment, differentiation media do not include one or both TPO and SCF. In one embodiment, differentiation media do not include TPO. In one embodiment, differentiation media do not include SCF.

If SCF is included in a differentiation medium, a concentration of SCF therein may range between about 0.5 ng/mL and 500 ng/mL, between about 1 ng/mL and 250 ng/mL, between about 5 ng/mL and 100 ng/mL, or between about 10 ng/mL and 50 ng/mL.

If FLT3L is included in a differentiation medium, a concentration of FLT3L therein may range between about 0.5 ng/mL and 500 ng/mL, between about 1 ng/mL and 250 ng/mL, between about 5 ng/mL and 100 ng/mL, or between about 10 ng/mL and 50 ng/mL.

If TPO is included in a differentiation medium, a concentration of TPO therein may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, differentiation media further comprise at least one other cytokine. In one embodiment, the at least one other cytokine is one or more interleukin. In one embodiment, the at least one other cytokine is one or more of IL-1, IL-2, IL-3, IL-4, IL-6, IL-7, IL-10, IL-15, IL-17, and IL-21. In one embodiment, the at least one other cytokine is IL-3, IL-6, or IL-7, or any combination thereof.

A concentration of the at least one other cytokine in a differentiation medium may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, differentiation media further comprise one or more additional cytokines or growth factors, or small molecules, to further enhance the differentiation of B lineage cells from a population of B cell precursors. Non-limiting examples of the one or more additional cytokines or growth factors that may be included in a differentiation medium include erythropoietin (EPO), insulin growth factor 1 (IGF-1) and insulin growth factor 2 (IGF-2), B cell activating factor (BAFF), a proliferation-inducing ligand (APRIL), and interferon gamma (IFN-g).

A concentration of the one or more additional cytokines or growth factors included in a differentiation medium may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, differentiation media may be formulated as a complete medium. In one embodiment, differentiation media may be prepared freshly before use, and thus the basal medium may be stored separately from one or more supplements to be added to the basal medium. In one example, the growth factors and cytokines may be combined in a supplement and added to the basal medium just prior to use of the complete differentiation medium in a derivation/differentiation method.

The growth factors and cytokines to be included in a differentiation medium may be sourced from various commercial suppliers, and may be recombinant.

Differentiation media of this disclosure may synergize with a substrate for supporting the culture of the population of B cell precursors (for the differentiation of B lineage cells). In one embodiment, stromal or feeder cells may be used together with cell culture media of this disclosure. Non-exhaustive examples of such cells include the embryonic liver cell line EL08.1D2, AFT024 cells, OP9 cells, MS-5 or M2-10B4 cells, mouse embryonic fibroblasts or stromal cells from embryonic aorta-gonad mesonephros.

In one embodiment, culturing the population of B cell precursors is done under feeder cell-free and/or stroma cell-free conditions. Such approaches may utilize medium previously conditioned by stromal/feeder cells, or such a system may utilize a stroma/feeder cell replacement. A stroma/feeder cell replacement may comprise one or more defined components that provide appropriate signals or attachment sites to cells in culture. Such components may be included in a differentiation medium or employed as a coating applied to an inner culture surface of a culture vessel or on solid surfaces suspended in a cell culture media, such as on particles, beads, microcarriers, or the like. Non-exhaustive examples of such components may include fibronectin coatings, gelatin coatings, collagen coatings, or Matrigel (Corning).

In one embodiment, culturing the population of B cell precursors is in the presence of an extracellular matrix protein or a cell adhesion molecule (while in the absence of feeder cell and/or stroma cell support). In one embodiment, the extracellular matrix protein or the cell adhesion molecule is solubilized in a differentiation medium or coated on a surface in contact with the differentiation medium. In one embodiment, the extracellular matrix protein is a fibronectin, a vitronectin, a laminin, ECM1, SPARC, or osteopontin. In one embodiment, the cell adhesion molecule is a vascular cell adhesion molecule (e.g. VCAM-1) or an immobilized SCF protein (e.g. SCF-Fc).

In one embodiment, different combinations of the foregoing proteins may be used. In one embodiment, combinations of extracellular matrix proteins are used. In one embodiment, combinations of cell adhesion molecules are used. In one embodiment, combinations of extracellular matrix protein(s) and cell adhesion molecule(s) are used.

Where included, a concentration of an extracellular matrix protein or a cell adhesion molecule that synergizes with a differentiation medium ranges between about 0.1 to 100 μg/mL (or 0.03 to 30 μg/well of a 96-well plate), between about 0.2 to 50 μg/mL (or 0.06 to 15 μg/well of a 96-well plate), or between about 0.5 to 20 μg/mL (or 0.15 to 6 μg/well of a 96-well plate).

In one embodiment, media of this disclosure are not dependent upon use together with an extracellular matrix protein or a cell adhesion molecule.

Culturing the population of B cell precursors in a differentiation medium may be for any period of time that does not impact their viability or capacity to differentiate to downstream lineages. In one embodiment, a method of differentiating a population of B cell precursors to a population of B lineage cells comprises culturing the population of B cell precursors in differentiation medium for between about 1 to 28 days, between about 3 and 25 days, between about 5 and 21 days, or between about 7 and 14 days. In one embodiment, a method of differentiating a population of B cell precursors to a population of B lineage cells comprises culturing the population of B cell precursors in differentiation medium for between about 3 to 14 days.

In one embodiment, 1% or more of cells differentiated using differentiation media are B lineage cells (e.g. express CD19). In one embodiment, 5% or more of cells differentiated using differentiation media are B lineage cells. In one embodiment, 10% or more of cells differentiated using differentiation media are B lineage cells. In one embodiment, 20% or more of cells differentiated using differentiation media are B lineage cells. In one embodiment, 30% or more of cells differentiated using differentiation media are B lineage cells. In one embodiment, 40% or more of cells differentiated using differentiation media are B lineage cells. In one embodiment, 50% or more of cells differentiated using differentiation media are B lineage cells. In one embodiment, 60% or more of cells differentiated using differentiation media are B lineage cells. In one embodiment, 70% or more of cells differentiated using differentiation media are B lineage cells. In one embodiment, 80% or more of cells differentiated using differentiation media are B lineage cells. In one embodiment, 90% or more of cells differentiated using differentiation media are B lineage cells.

In one embodiment, at least a fraction of the CD19⁺ B lineage cells (differentiated using differentiation media) are IgM⁺ cells. In one embodiment, about 1% or more CD19⁺ B lineage cells are IgM⁺ cells. In one embodiment, about 2% or more CD19⁺ B lineage cells are IgM⁺ cells. In one embodiment, about 3% or more CD19⁺ B lineage cells are IgM⁺ cells. In one embodiment, about 4% or more CD19⁺ B lineage cells are IgM⁺ cells. In one embodiment, about 5% or more CD19⁺ B lineage cells are IgM⁺ cells. In one embodiment, about 10% or more CD19⁺ B lineage cells are IgM⁺ cells.

In one embodiment, differentiation of B lineage cells using a differentiation medium yields 10 or more CD19⁺ cells per input cell, 25 or more CD19⁺ cells per input cell, 50 or more CD19⁺ cells per input cell, 100 or more CD19⁺ cells per input cell, 250 or more CD19⁺ cells per input cell, 500 or more CD19⁺ cells per input cell, 1000 or more CD19⁺ cells per input cell, or 2000 or more CD19⁺ cells per input cell. Further, in one embodiment, differentiation of B lineage cells using a differentiation medium yields 1 or more IgM⁺ cells per input cell, 5 or more IgM⁺ cells per input cell, 10 or more IgM⁺ cells per input cell, 20 or more IgM⁺ cells per input cell, 50 or more IgM⁺ cells per input cell, or 100 or more IgM⁺ cells per input cell.

In another aspect, directed differentiation methods of this disclosure comprise contacting a population of B lineage cells with a downstream differentiation medium, and culturing the population of B lineage cells in the downstream differentiation medium for a time sufficient to obtain and/or expand IgM⁺ cells (and/or antibody-secreting cells).

In one embodiment, the population of B lineage cells comprise, or are a population of, CD19⁺ cells, such as double positive CD10⁺CD19⁺ B lineage cells or single positive CD19⁺ cells.

In one embodiment, the methods further comprise obtaining more IgM⁺ cells after culturing in a downstream differentiation medium than are included among the cells output after culturing in a differentiation medium of this disclosure (e.g. the population of B lineage cells), or among the population of CD19⁺ cells (e.g. double positive CD10⁺CD19⁺ B lineage cells).

A downstream differentiation medium (which may also be considered a differentiation and expansion medium of IgM⁺ cells) is any medium that may be used to differentiate a population of B lineage cells (to a population of IgM⁺ cells) or expand IgM⁺ cells. In a preferred embodiment, the downstream differentiation medium is serum-free. If downstream differentiation media are serum-free, it may be necessary to include in such media a serum replacement supplement, such as BIT 9500 Serum Substitute (STEMCELL Technologies, Catalogue #09500), or other commercially available serum replacement solutions. Alternatively, components ordinarily present in serum needed to culture or differentiate any cells of this disclosure may be individually added at acceptable concentrations into a downstream differentiation medium. In one embodiment, a component ordinarily present in serum is albumin. If an albumin is included in a downstream differentiation medium (in place of serum), it may be from any species, but is typically either bovine or human. In some embodiments, an albumin may be recombinant.

Downstream differentiation media of this disclosure, alternatively referred to as “DDM” media, will include a basal medium formulated as appropriate to culture HSPC and to support derivation of B cell precursors, differentiation of B lineage cells, including IgM⁺ cells. Thus, a suitable basal medium is any basal medium that is supportive of culturing cells of the hematopoietic lineage, and in particular B lineage cells. Exemplary basal media include, but are not limited to, STEMdiff™ Hematopoietic-EB Basal Medium (STEMCELL Technologies, Catalogue #100-171), STEMdiff™ Hematopoietic Basal Medium (STEMCELL Technologies, Catalogue #05311), STEMdiff™ APEL™2 Medium (STEMCELL Technologies, Catalogue #05270), StemSpan™ AOF Medium (STEMCELL Technologies, Catalogue #100-0130), StemSpan™ SFEM & SFEM II, ImmunoCult™ XF (STEMCELL Technologies, Catalogue #09650, 09655, 10981), or any other commercially available basal medium fit for the purpose. Common components used to formulate such basal media may include salts, buffers, lipids, amino acids, trace elements, certain proteins, vitamins, minerals, reducing agents, etc. In one embodiment, basal media are formulated to optimally support the differentiation of HSPC to B lineage cells, including IgM⁺ cells, therefrom.

In one embodiment, downstream differentiation media comprise a ligand of human CD40. A ligand of human CD40 may be isolated and/or used in native form. Alternatively, a ligand of human CD40 may be engineered for increased activity and/or half-life and/or stability. Whether native or engineered, the ligand of CD40 may be procured from a commercial supplier, and may be recombinant.

If included, a ligand of CD40 in a downstream differentiation may be present at a concentration ranging between about 10 ng/mL and 5 μg/mL, between about 25 ng/mL and 2 μg/mL, between about 50 ng/mL and 1 μg/mL, or between about 100 ng/mL and 500 ng/mL.

In one embodiment, a downstream differentiation medium further comprises at least one other cytokine. In one embodiment, the at least one other cytokine is one or more interleukin. In one embodiment, the at least one other cytokine is one or more of IL-1, IL-2, IL-3, IL-4, IL-6, IL-7, IL-10, IL-15, IL-17, and IL-21. In one embodiment, the at least one other cytokine is one or more of IL-2, IL-4, IL-6, IL-7, IL-10, or IL-21, or any combination thereof.

A concentration of the at least one other cytokine (or each of the at least one other cytokine) in a downstream differentiation medium may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, downstream differentiation media may further comprise one or more additional cytokines or growth factors, or small molecules, to further enhance the differentiation of IgM⁺ cells and antibody secreting cells from a population of B lineage cells. Non-limiting examples of the one or more additional cytokines or growth factors that may be included in a differentiation medium include erythropoietin (EPO), insulin growth factor 1 (IGF-1), insulin growth factor 2 (IGF-2), B cell activating factor (BAFF), a proliferation-inducing ligand (APRIL), and interferon gamma (IFN-g).

A concentration of the one or more additional growth factors included in a downstream differentiation medium may range between about 0.1 ng/mL and 1 μg/mL, between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, the one or more additional growth factors comprised in a downstream differentiation medium may be selected from one or more of SCF, TPO, and FLT3L. In one embodiment, downstream differentiation media may comprise two or more of SCF, TPO, and FLT3L. In one embodiment, downstream differentiation media may comprise each or none of SCF, TPO, and FLT3L. In one embodiment, a downstream differentiation medium does not include one or both of SCF and FLT3L. In one embodiment, a downstream differentiation medium does not include TPO, and does not include one or both of SCF and FLT3L.

If SCF is included in a downstream differentiation medium, a concentration of SCF therein may range between about 0.5 ng/mL and 500 ng/mL, between about 1 ng/mL and 250 ng/mL, between about 5 ng/mL and 100 ng/mL, or between about 10 ng/mL and 50 ng/mL.

If FLT3L is included in a downstream differentiation medium, a concentration of FLT3L therein may range between about 0.5 ng/mL and 500 ng/mL, between about 1 ng/mL and 250 ng/mL, between about 5 ng/mL and 100 ng/mL, or between about 10 ng/mL and 50 ng/mL.

If TPO is included in a downstream differentiation medium, a concentration of TPO therein may range between about 0.5 ng/mL and 500 ng/mL, between about 1 ng/mL and 250 ng/mL, between about 5 ng/mL and 100 ng/mL, or between about 10 ng/mL and 50 ng/mL.

In one embodiment, downstream differentiation media comprise a basal medium and one or both of a ligand of CD40 and at least one other cytokine. In one embodiment, downstream differentiation media comprise a basal medium and one or more of a ligand of CD40, at least one other cytokine, and at least one additional cytokine. In one embodiment, downstream differentiation media comprise a basal medium one or both of a ligand of CD40 and at least one additional cytokine.

In one embodiment, a downstream differentiation medium may be formulated as a complete medium. In one embodiment, a downstream differentiation medium may be prepared freshly before use, and thus the basal medium may be stored separately from one or more supplements to be added to the basal medium. In one example, the growth factors and cytokines may be combined in a supplement and added to the basal medium just prior to use of the complete downstream differentiation medium in a derivation/differentiation method.

The growth factors and cytokines to be included in a downstream differentiation medium may be sourced from various commercial suppliers, and may be recombinant.

Downstream differentiation media of this disclosure may synergize with a substrate for supporting the culture of the population of B lineage cells. In one embodiment, stromal or feeder cells may be used together with cell culture media of this disclosure. Non-exhaustive examples of such cells include the embryonic liver cell line EL08.1D2, AFT024 cells, OP9 cells, MS-5 or M2-10B4 cells, mouse embryonic fibroblasts or stromal cells from embryonic aorta-gonad mesonephros (AGM).

In one embodiment, culturing the population of B lineage cells is done under feeder cell-free and/or stroma cell-free conditions. Such approaches may utilize medium previously conditioned by stromal/feeder cells, or such a system may utilize a stroma/feeder cell replacement. A stroma/feeder cell replacement may comprise one or more defined components that provide appropriate signals or attachment sites to cells in culture. Such components may be included in a downstream differentiation medium or employed as a coating applied to an inner culture surface of a culture vessel or on solid surfaces suspended in a cell culture media, such as on particles, beads, microcarriers, or the like. Non-exhaustive examples of such components may include fibronectin coatings, gelatin coatings, collagen coatings, or Matrigel (Corning).

In one embodiment, culturing the population of B lineage cells is in the presence of an extracellular matrix protein or a cell adhesion molecule (while in the absence of feeder cell and/or stroma cell support). In one embodiment, the extracellular matrix protein or the cell adhesion molecule is solubilized in a downstream differentiation medium or coated on a surface in contact with the downstream differentiation medium. In one embodiment, the extracellular matrix protein is a fibronectin, a vitronectin, a laminin, ECM1, SPARC, or osteopontin. In one embodiment, the cell adhesion molecule is a vascular cell adhesion molecule (e.g. VCAM-1) or an immobilized SCF protein (e.g. SCF-Fc).

In one embodiment, a combination of extracellular matrix proteins and cell adhesions molecules are used. In one embodiment, combinations of extracellular matrix proteins are used. In one embodiment, combinations of cell adhesion molecules are used. In one embodiment, combinations of extracellular matrix protein(s) and cell adhesion molecule(s) are used.

Where included, a concentration of an extracellular matrix protein or a cell adhesion molecule that synergizes with a downstream differentiation medium ranges between about 0.1 to 100 μg/mL (or 0.03 to 30 μg/well of a 96-well plate), between about 0.2 to 50 μg/mL (or 0.06 to 15 μg/well of a 96-well plate), or between about 0.5 to 20 μg/mL (or 0.15 to 6 μg/well of a 96-well plate).

In one embodiment, media of this disclosure are not dependent upon use together with an extracellular matrix protein or a cell adhesion molecule.

Culturing the population of B lineage cells in a downstream differentiation medium may be for any period of time that does not impact their viability or capacity to differentiate to downstream lineages. In one embodiment, a method of differentiating a population of B lineage cells to a population of IgM⁺ cells and/or antibody secreting cells comprises culturing the population of B lineage cells in a downstream differentiation medium for between about 1 to 28 days, between about 3 and 25 days, between about 5 and 21 days, or between about 7 and 14 days. In one embodiment, a method of differentiating a population of B lineage cells to a population of IgM⁺ cells comprises culturing the population of B lineage cells in a downstream differentiation medium for between about 3 to 21 days.

In one embodiment, 10% or more of the cells after culturing B lineage cells in a downstream differentiation medium are CD19⁺ cells. In one embodiment, 20% or more of the cells after culturing B lineage cells in a downstream differentiation medium are CD19⁺ cells. In one embodiment, 30% or more of the cells after culturing B lineage cells in a downstream differentiation medium are CD19⁺ cells. In one embodiment, 40% or more of the cells after culturing B lineage cells in a downstream differentiation medium are CD19⁺ cells. In one embodiment, 50% or more of the cells after culturing B lineage cells in a downstream differentiation medium are CD19⁺ cells. In one embodiment, 60% or more of the cells after culturing B lineage cells in a downstream differentiation medium are CD19⁺ cells. In one embodiment, 70% or more of the cells after culturing B lineage cells in a downstream differentiation medium are CD19⁺ cells. In one embodiment, 80% or more of the cells after culturing B lineage cells in a downstream differentiation medium are CD19⁺ cells. In one embodiment, 90% or more of the cells after culturing B lineage cells in a downstream differentiation medium are CD19⁺ cells.

In one embodiment, 1% of the CD19⁺ cells after culturing B lineage cells in a downstream differentiation medium are IgM⁺ cells. In one embodiment, 2% of the CD19⁺ cells after culturing B lineage cells in a downstream differentiation medium are IgM⁺ cells. In one embodiment, 3% of the CD19⁺ cells after culturing B lineage cells in a downstream differentiation medium are IgM⁺ cells. In one embodiment, 4% of the CD19⁺ cells after culturing B lineage cells in a downstream differentiation medium are IgM⁺ cells. In one embodiment, 5% or more of the CD19⁺ cells after culturing B lineage cells in a downstream differentiation medium are IgM⁺ cells. In one embodiment, 10% or more of the CD19⁺ cells after culturing B lineage cells in a downstream differentiation medium are IgM⁺ cells. In one embodiment, 15% or more of the CD19⁺ cells after culturing B lineage cells in a downstream differentiation medium are IgM⁺ cells. In one embodiment, 20% or more of the CD19⁺ cells after culturing B lineage cells in a downstream differentiation medium are IgM⁺ cells.

In one embodiment, downstream differentiation of B lineage cells using a downstream differentiation medium yields 50 or more CD19⁺ cells per input cell, 100 or more CD19⁺ cells per input cell, 250 or more CD19⁺ cells per input cell, 500 or more CD19⁺ cells per input cell, 1000 or more CD19⁺ cells per input cell, 2000 or more CD19⁺ cells per input cell, 3000 or more CD19⁺ cells per input cell, or 4000 or more CD19⁺ cells per input cell. Further, in one embodiment, downstream differentiation of B lineage cells using a downstream differentiation medium yields 10 or more IgM⁺ cells per input cell, 25 or more IgM⁺ cells per input cell, 50 or more IgM⁺ cells per input cell, 100 or more IgM⁺ cells per input cell, 150 or more IgM⁺ cells per input cell, or 200 or more IgM⁺ cells per input cell.

In one embodiment, following culture in a downstream differentiation medium about 0.5% or more of the output cells are antibody secreting cells. In one embodiment, about 1% or more of the output cells are antibody secreting cells. In one embodiment, about 2% or more of the output cells are antibody secreting cells. In one embodiment, about 3% or more of the output cells are antibody secreting cells. In one embodiment, about 4% or more of the output cells are antibody secreting cells. In one embodiment, about 5% or more of the output cells are antibody secreting cells. In one embodiment, about 10% or more of the output cells are antibody secreting cells.

In one embodiment, the antibody secreting cells are CD19⁺. In one embodiment, the antibody secreting cells are CD19−, and such a population of antibody secreting cells may be CD138⁺. In one embodiment, the antibody secreting cells are IgM⁻. In one embodiment, the antibody secreting cells secrete either IgM or IgG.

In embodiments of the methods disclosed herein where the HSPC are PSC-derived, the PSC may be cultured under serum-free conditions. In the same or different embodiment, the PSC may be cultured under stromal cell-free conditions and/or feeder-free conditions. Differentiating CD34⁺ HSPC from PSC may be done using a commercially available kit, such as the STEMdiff™ Hematopoietic Kit (STEMCELL Technologies) or the STEMdiff™ Hematopoietic-EB Basal Medium, together with EB Supplement A and EB Supplement B (STEMCELL Technologies). In this step, or at any step of this methods disclosed herein, it may be desirable to purify/enrich the cells of interest before proceeding to the next step of the methods. Ways to purify/enrich cells are known, such as through immunomagnetic cell separation or fluorescence activated cell sorting.

IgM⁺ B cells may further mature into IgM⁺IgD⁺ naïve B cells (via IgM⁺IgD⁺ transitional B cell stages), IgM antibody secreting cells, or upon isotype switching to either IgG⁺, IgE⁺, or IgA⁺ memory B cells or plasma cells. Such memory B cells may also differentiate to antibody secreting cells (able to secrete IgM, IgG, IgE or IgA).

In one embodiment, a method of deriving a population of B cell precursors from a population of PSC-derived HSPC may comprise forming the PSC into aggregates prior to differentiating the PSC to HSPC, as described above, and then subjecting such PSC-derived HSPC to derivation medium conditions.

PSC may be formed into aggregates using any known approach. For example, aggregates of PSC may be formed by depositing a desired number of PSC into the bottom of a tube or a well of a cell culture plate. Or, aggregates may be formed by depositing a desired number of PSC into a well of an Aggrewell™ microwell device (STEMCELL Technologies), to ensure the efficient and reproducible formation of uniformly sized aggregates of PSC.

In one embodiment, the number of PSC used to form the aggregates is between about 1 and 100,000. In one embodiment, the number of PSC used to form the aggregates is between about 10 and 10,000. In one embodiment, the number of PSC used to form the aggregates is between about 100 and 1,000.

Therefore, in one embodiment, the aggregates of PSC may be formed in a microwell device. In one embodiment, the aggregates of PSC are formed from about 1000 cells or about 500 cells.

In addition to disclosing media for and methods of i) enriching HSPC, ii) differentiating PSC to mesoderm precursors, iii) deriving a population of B cell precursors from HSPC, whether PSC- or tissue-derived, iv) differentiating B lineage cells from B cell precursors, v) differentiating IgM⁺ and/or IgM-secreting cells from B lineage cells, or vi) differentiating IgG⁺ and/or IgG-secreting cells from B lineage cells or a later stage cell type, the various media disclosed herein may be included in a system or a kit for the stepwise differentiation of PSC or tissue-derived HSPC through to immature or mature B cells, whether or not under serum- and/or feeder cell-free or stromal cell-free conditions.

In some embodiments, the entire system is performed under serum- and/or feeder cell-free or stromal cell-free conditions. In some embodiments, only certain aspects of the system are performed under serum- and/or feeder cell-free or stromal cell-free conditions. For example, but without limiting the generality of the foregoing, differentiating PSC to mesoderm precursors, PSC-derived mesoderm precursors to hematopoietic progenitor cells, PSC-derived hematopoietic progenitors to lymphoid progenitors (e.g. a population of B cell precursors), differentiating B cell precursors to B lineage cells, and differentiating B lineage cells to either IgM-expressing (and/or IgM-secreting cells) and/or IgG-expressing (and/or IgG-secreting cells) are performed under serum- and/or feeder cell-free or stromal cell-free conditions, while further downstream stages may or may not. Conversely, earlier stages of the system may or may not be performed under serum- and/or feeder cell-free or stromal-cell free conditions, while subsequent stages are performed under serum- and/or stroma-free conditions.

In one embodiment, such system or kit may include in the context of a PSC workflow one, two, three, four, five, six, seven or more of the following components: a first culture system for differentiating PSC to mesoderm precursors; a second culture system for differentiating PSC-derived mesoderm precursors to hematopoietic progenitors cells; a third culture system for differentiating PSC-derived hematopoietic progenitors cells to one or more subsets of lymphoid progenitor cells; a fourth culture system for differentiating PSC-derived lymphoid progenitor (e.g. population of B cell precursors); a fifth culture system for differentiating PSC-derived lymphoid progenitor cells (e.g. population of B cell precursors) to a population of B lineage cells; a sixth culture system for differentiating a population of B lineage cells to IgM-expressing (and/or IgM-secreting cells) and/or IgG-expressing (and/or IgG-secreting cells); a coating substrate; a first kit for enriching, either positively or negatively, a population of HSPC; a second kit for enriching, either positively or negatively, a population of B cell precursors; a third kit for enriching, either positively or negatively, a population of B lineage cells; a fourth kit for enriching, either positively or negatively, a population of immature B cells; and a fifth kit for enriching, either positively or negatively, a population of mature B cells.

In one embodiment, such system or kit may include in the context of a primary CD34⁺ cell or tissue derived CD34⁺ cell workflow one, two, three, four, five, six, seven or more of the following components: a first culture system for deriving B cell precursors from a population of CD34⁺ HSPC; a second culture system for differentiating B lineage cells from a population of B cell precursors; a third culture system for further differentiating IgM⁺ or IgM-secreting cells from a population of B lineage cells; a coating substrate; a first kit for enriching, either positively or negatively, a population of HSPC; a second kit for enriching, either positively or negatively, a population of B cell precursors; a third kit for enriching, either positively or negatively, a population of B lineage cells; a fourth kit for enriching, either positively or negatively, a population of immature B cells; and a fifth kit for enriching, either positively or negatively, a population of mature B cells.

Cells obtained with the media disclosed herein or by the methods disclosed herein, may be used for any downstream assay or purpose. In one embodiment, the cells of this disclosure (whether the population of B cell precursors, the population of CD19⁺ or CD10⁺CD19⁺ B lineage cells, or the IgM⁺ or IgM secreting cells) may be used in research applications to study the biology of B cell development or of B cell disease, such as cancer.

In one embodiment, the cells of this disclosure may be used for transplantation purposes into a patient in need, such as a patient suffering from a hematopoietic (e.g. a B cell) disorder. In such embodiments, the cells to be transplanted into the patient in need may be the population of B cell precursors, the population of CD19⁺ or CD10⁺CD19⁺ B lineage cells, or the IgM⁺ or Ig secreting cells, such as IgM and/or IgG. Such cells may be edited using known gene editing technology to either introduce one or more transgenes, remove one or more fragments of DNA, or to create one or more hypo- or hypermorphic mutations. In one embodiment, the cells to be transplanted are PSC-derived, and are thus a potentially universal source of allogeneic cells. In one embodiment, the cells to be transplanted are tissue-derived, and are thus potentially a source of autologous cells.

In one embodiment, the cells of this disclosure may be used to assess their responsiveness to test conditions, such as in toxicity studies or drug screens. In such embodiments, the originating cells may correspond to a non-diseased or a diseased state. In some embodiments, the diseased state may be introduced into a founding cell or cells, such as by gene editing technology.

The following non-limiting examples are illustrative of the present disclosure.

### EXAMPLES

### Example 1: Enriching CD34⁺ HSPC from Donor Samples

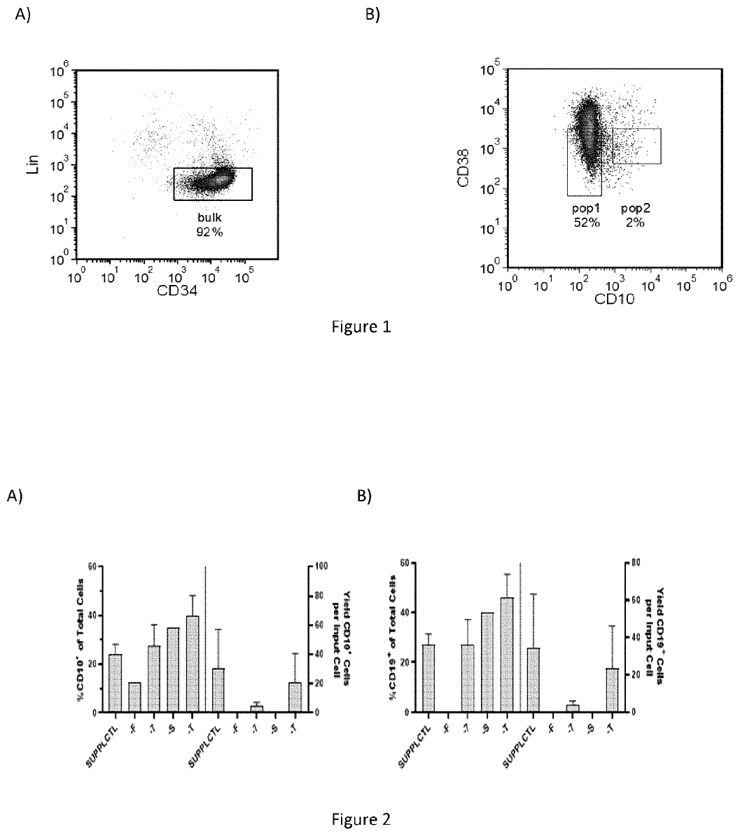

Cord blood units were procured from commercial suppliers and CD34⁺ HSPC were enriched using the EasySep Human Cord Blood CD34 Positive Selection Kit II (STEMCELL Technologies), and then either frozen down in serum with 10% DMSO or used fresh. Following, CD34⁺ cells were stained and then sorted (FACS Aria) for Lin⁻ (Lin included CD3, CD14, CD15, CD16, CD19, CD56, and CD66b), CD34⁺CD38^{-/mid/low}CD10⁻ cells, termed “pop1”. A second, more definitive, sorted population used as a control was Lin⁻CD34⁺CD38^{mid}CD10⁺, termed “pop2” (FIG. 1).

### Example 2: Flow Cytometry and Determination of Cell Count and Yield

At any stage of the directed differentiation protocols outlined in this disclosure, the samples can be harvested and the phenotypes thereof can be assessed by flow cytometry. The following general protocol equally applies to measuring CD34, CD10, CD19, CD20 and IgM.

Briefly, the cell sample was harvested by centrifugation and appropriately washed. The cell sample was then stained with fluorophore-conjugated antibodies against the antigen of choice and analyzed on the CytoFLEX S™ flow cytometer (Beckman-Coulter). Dead cells were excluded by light scatter profile and DRAQ7 staining.

Total viable cell counts were obtained using the NucleoCounter NC250 (Chemometec) in accordance with the manufacturer's recommendations. Cells were diluted, as required, prior to staining with a mixture of acridine orange and DAPI (AO/DAPI). In this mixture, AO labels the cell membrane and DAPI labels nucleic acid in dead/dying cells—together enabling photographic discrimination of viable vs. non-viable cells in the sample. The NC250 software then analyzes the resulting images and reports the cell counts, including viable cell concentration. To calculate the yield of particular cells per input cell, total viable counts were multiplied by the % frequency of the given cell type. For example, to calculate the yield of CD10⁺ cells per input CD34⁺ cell, the viable cell count is first multiplied by the % CD10⁺ obtained by flow cytometry. This number is then divided by the number of input cells (in the case input CD34⁺ cells) to obtain the final value. Input CD34⁺ cell numbers were obtained by multiplying total cells cultured in one well by frequency of CD34⁺ cells after cell separation.

### Example 3: Deriving B Cell Precursors from Cord Blood-Derived CD34⁺ HSPC

The enriched CD34⁺ HSPC of Example 1 were differentiated to B cell precursors in a derivation medium. Derivation media typically comprised a basal medium, such as StemSpan™ SFEMII (STEMCELL Technologies, Catalogue #09655), and an assortment of stage-specific cytokines and growth factors. An initial iteration of a derivation medium comprised SCF, TPO, FLT3L, and IL-7, and experiments were carried out testing the different combinations of such factors (FIGS. 2, 3 and 6).

Briefly, pop2 cells were sorted from an enriched population of CD34⁺ HSPC, essentially as described in Example 1, and 5000 cells were seeded/well of a 24-well plate into the tested derivation media formulations. After 14 days in culture in the different media formulations, the output cells were analyzed by flow cytometry for CD10⁺ (FIG. 2A) and CD19⁺ (FIG. 2B) cell frequency and yield.

It was observed that TPO was dispensable in a derivation medium for deriving B cell precursors from cord blood-derived CD34⁺ HSPC. Having identified that TPO was dispensable in the derivation medium, the effect of other cytokines (e.g. IL-3 and/or IL-6) added into such a derivation medium was tested.

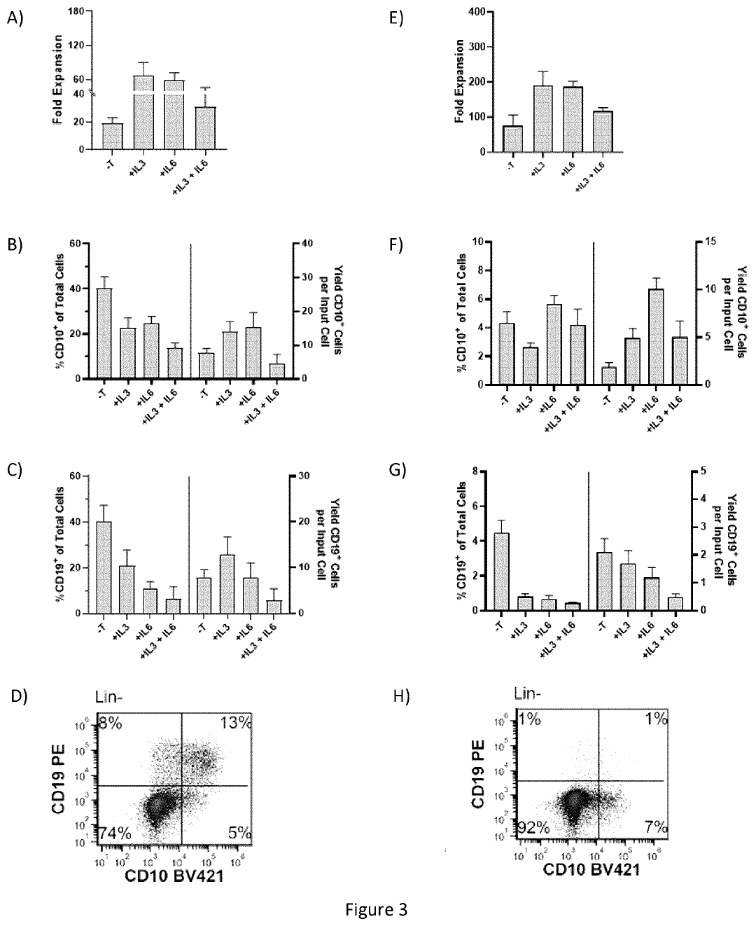

Briefly, pop2 cells were sorted from an enriched population of CD34⁺ HSPC, seeded, and cultured, essentially as described above. After 14 days in culture in the different media formulations, the output cells were analyzed by flow cytometry for total fold expansion (FIG. 3A), CD10⁺ cell frequency and yield (FIG. 3B) and CD19⁺ cell frequency and yield (FIG. 3C). An exemplary plot of day 14 cells analyzed by flow cytometry is shown in FIG. 3D.

It was observed that inclusion of either IL-3 or IL-6 alone, but not in combination, in a derivation medium improved the yield of CD10⁺ cells from pop2 cells, and that IL-3 alone improved yield of CD19⁺ cells from pop2 cells.

Similar experiments to those carried out and reported above were done using pop1 cells, which were enriched, sorted and seeded as described in Example 1. After 14 days in culture in the different media formulations, the output cells were analyzed by flow cytometry for total fold expansion (FIG. 3E), CD10⁺ cell frequency and yield (FIG. 3F), and CD19⁺ cell frequency and yield (FIG. 3G). An exemplary plot of day 14 cells analyzed by flow cytometry is shown in FIG. 3H.

It was observed that inclusion of IL-3 or IL-6, either alone or in combination, after 14 days in a derivation medium improved the yield of CD10⁺ cells from pop1 cells.

### Example 4: Differentiating CD19⁺ B Lineage Cells from Cord Blood Derived B Cell Precursors

B cell precursors were derived from CD34⁺ HSPC (i.e. pop2 cells) essentially as described in Example 3, and differentiated into CD19⁺ B lineage cells, as described below.

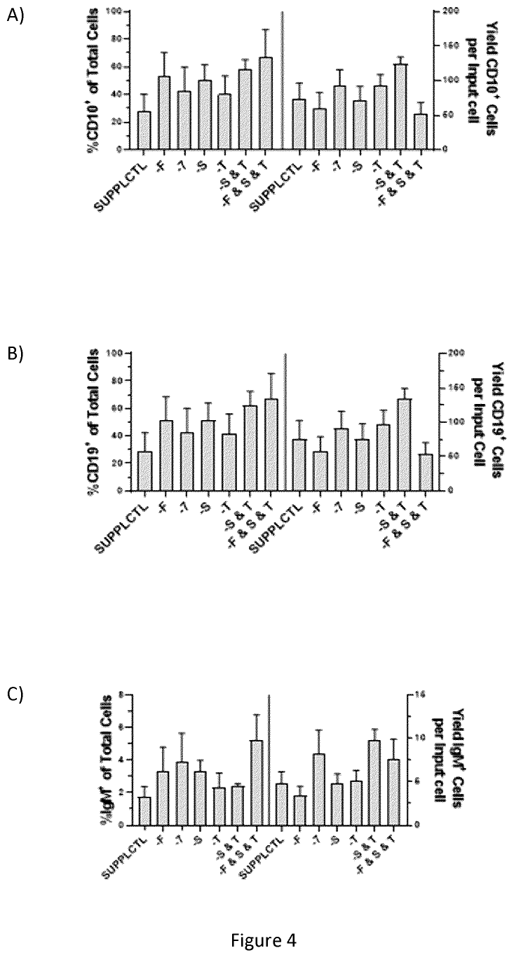

In an initial experiment, pop2 cells were cultured for 14 days in a control derivation medium (e.g. SUPPLCTL). After 14 days, the cells were transitioned into various differentiation medium formulations and cultured for an additional 14 days. The tested differentiation medium conditions were formulated to lack one or more of SCF, TPO, IL-7, or FLT3L, as indicated in FIG. 4. Day 28 cells were analyzed by flow cytometry for frequency and yield of CD10⁺ cells (FIG. 4A), of CD19⁺ B lineage cells (FIG. 4B), and of IgM⁺ cells as a function of CD19⁺ cells (FIG. 4C).

It was observed that all differentiation medium formulations resulted in appreciable frequency and yield across each cell population analyzed.

In a follow-up experiment, a derivation medium as described in Example 3 was used for 14 days to derive B cell precursors from pop2 cells. After 14 days, the output cells were harvested, counted, and analyzed by flow cytometry for expression of CD10 and CD19, and re-seeded at 1-2×10⁵ cells/well of a 24-well plate into differentiation medium as described in the paragraphs above. After two weeks in culture, all conditions were harvested, counted, and analyzed by flow cytometry for expression of CD10, CD19 and IgM (data not shown). It was observed that inclusion of IL-6 in a derivation medium synergized with the differentiation medium to yield the highest number of CD19⁺ B lineage cells (data not shown).

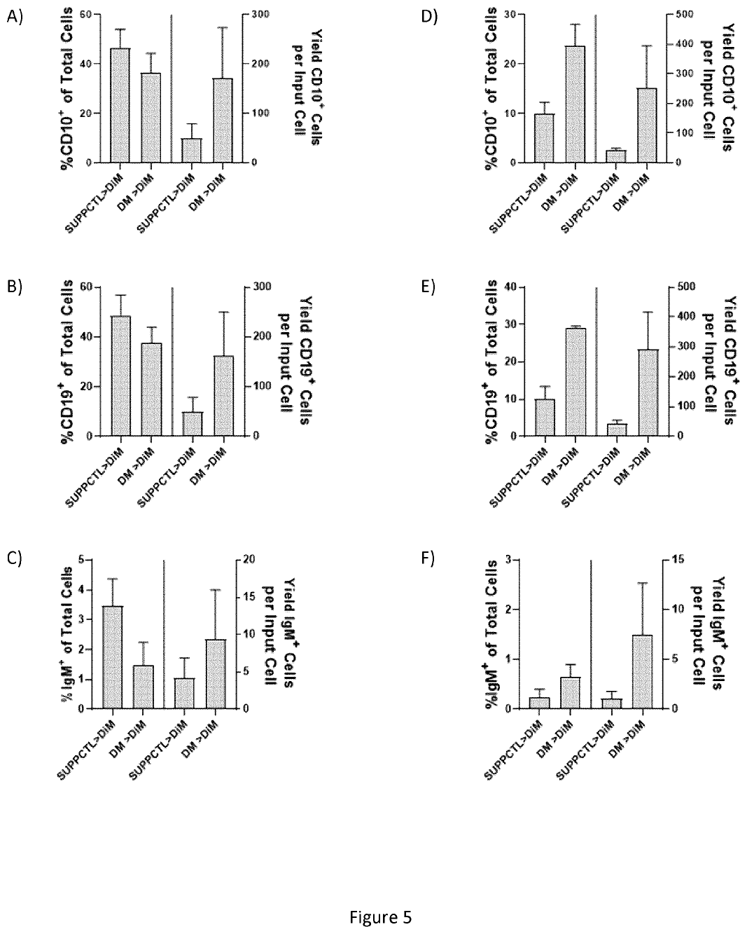

To confirm the above findings, pop2 cells were cultured for 14 days in a derivation medium formulation (as described in Example 3 and in this example), then the output cells were harvested, counted, and analyzed by flow cytometry for expression of markers such as CD10 and CD19. The day 14 cells were cultured a further 14 days in a differentiation medium formulation (as described above in this example), then harvested, counted, and analyzed by flow cytometry for expression (and frequency and yield) of CD10 (FIG. 5A), CD19 (FIG. 5B), and IgM (FIG. 5C). Pop1 cells were likewise cultured and analyzed by flow cytometry for expression (and frequency and yield) of CD10 (FIG. 5D), CD19 (FIG. 5E), and IgM (FIG. 5F).

For pop2 cells, the developed derivation and differentiation protocol could generate robust yields of approximately 150 CD19⁺ B lineage cells per input cell. Further, approximately 10 IgM⁺ cells could be generated per input cell. Considering that only 5% of enriched CD34⁺ HSPC correspond to pop2 cells, these efficiencies in serum- and feeder cell-free conditions are striking. As was observed in the context of pop2 cells but starting from pop1 cells, the developed derivation and differentiation protocol could generate robust yields of approximately 300 CD19⁺ B lineage cells per input cell (FIG. 5E). Further, >5 IgM⁺ cells could be generated per input cell (FIG. 5F).

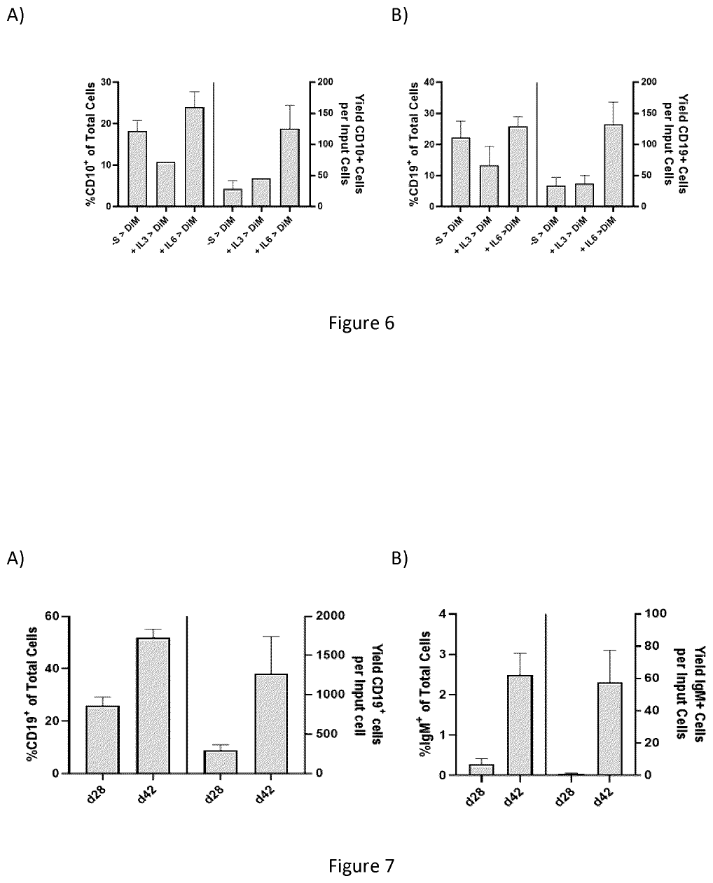

Similar to experiments carried out and reported above in this example, pop1 cells were enriched and isolated, as described in Example 1, then seeded at 5000 cells/well of a 24-well plate in a derivation medium formulation lacking SCF but including either IL-3 or IL-6, alone or in combination. After 14 days, the output cells were harvested, counted, and analyzed by flow cytometry for expression of CD10 and CD19, and re-seeded at 1-2×10⁵ cells/well into differentiation medium (as described above in this Example). After 14 days in culture, all conditions were harvested, counted, and analyzed by flow cytometry for expression of CD10, CD19 and IgM (data not shown). As above, day 28 cells were harvested, counted, and analyzed by flow cytometry for expression of CD10 (FIG. 6A) and CD19 (FIG. 6B).

As was observed in the context of pop2 cells, but in this case starting from pop1 cells, inclusion of IL-6 in a different derivation medium formulation synergized with the differentiation medium to yield the highest frequency and yield of CD19⁺ B lineage cells.

In an effort to increase the frequency and yield of CD19⁺ B lineage cells and IgM⁺ cells the duration of culture in the differentiation medium was extended from 14 days to 28 days. After the 42 day protocol (14 days in derivation medium and 28 days in differentiation medium), frequency and yield of pop1-derived CD19⁺ B lineage cells (FIG. 7A) and frequency of IgM⁺ cells among the CD19+ cells (FIG. 7B) were markedly increased. While pop1 cells are more prevalent than pop2 cells in the enriched population of CD34⁺ HSPC, the results achieved using serum- and feeder cell-free conditions are nevertheless striking.

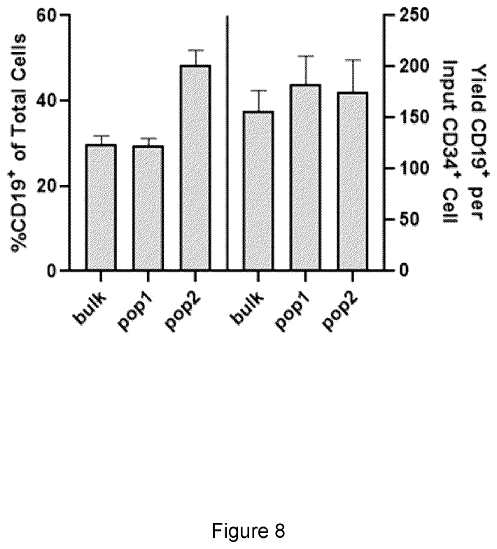

For each starting population of cells (bulk CD34⁺, pop1 and pop2), an overall summary of the frequency and yield of CD19⁺ B lineage cells is shown in FIG. 8. Each population of cells was separately subjected to derivation medium conditions as described in Example 3 and in this Example for 14 days, and then transitioned into differentiation medium conditions for 14 days as described in this Example. It is clear that marked frequencies and yields of CD19+ cells can be achieved using the media and practicing the methods of this disclosure regardless of the starting population of CD34⁺ cells.

### Example 5: Optimizing Downstream Differentiation of Cord Blood Derived CD19⁺ B Lineage Cells to IgM⁺ Cells

While the output of IgM⁺ cells could be improved with extended culture duration in a differentiation medium, other ways of enhancing IgM⁺ cell output were explored. Specifically, the effects of culture media additives were investigated for enhancing IgM⁺ cell output.

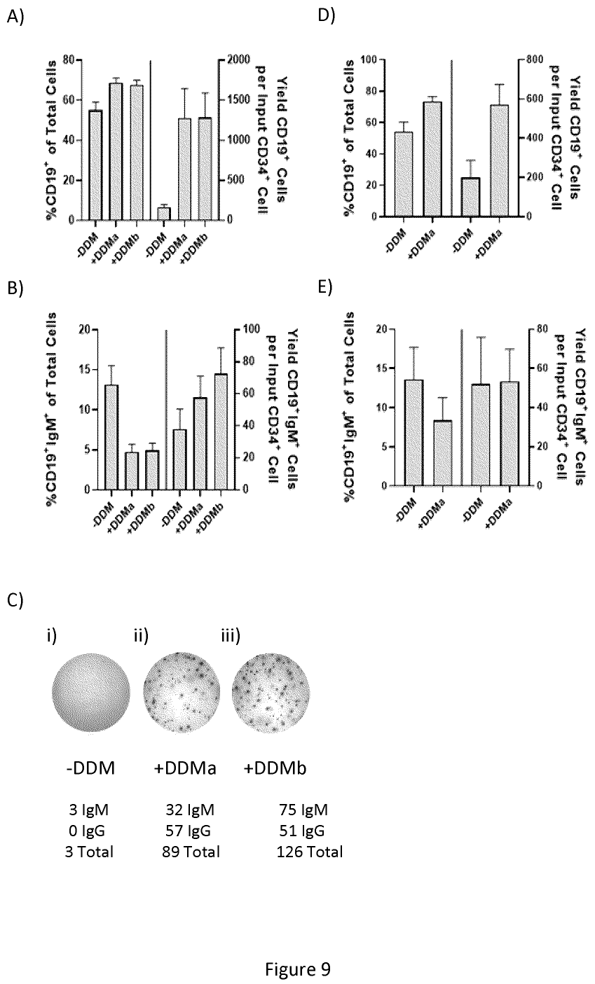

Briefly, 5000 pop2 cells were seeded/well of a 24-well plate into a derivation medium formulation (as in FIG. 3), after 14 days in culture the output cells were re-seeded at 1-2×10⁵ cells/well of a 24-well plate into differentiation medium (as in FIGS. 4 and 5) for an additional 14 days. After 28 days in culture, the output cells were re-seeded at 1-2×10⁵ cells/well in a 24-well plate and cultured for 7 days in various downstream differentiation media: basal SFEM II medium (STEMCELL Technologies) without cytokines or growth factors (“-DDM”); SFEMII (STEMCELL Technologies) basal medium supplemented with CD40L and a combination of cytokines (“DDMa”); and differentiation medium supplemented with CD40L and a combination of cytokines (“DDMb”). After the 35-day protocol, the output cells were harvested, counted, and analyzed by flow cytometry for expression of CD19 (FIG. 9A) and IgM (FIG. 9B). Secretion of IgM and IgG by the day-35 output cells was confirmed by ELISPOT (ImmunoSpot Human IgM/IgG-B-Cell Specific Double-Color ELISPOT kit). In comparison to a StemSpan™ SFEMII basal medium control (FIG. 9Ci), increased numbers of both IgM (red/grey dots) and IgG (blue/black dots) were detected in the CD40L-supplemented downstream differentiation media (FIGS. 9Cii and 9Ciii). Indeed, an output of CD19 and IgM expressing cells (in terms of yield) was achieved in CD40L-containing conditions.

As for pop2 cells, the effects of culture media additives for differentiating IgM⁺ cells beginning from bulk cells was investigated. Bulk cells were seeded, cultured, and analyzed as described for pop2 cells (FIGS. 9D and 9E). As was observed for pop2 cells, bulk cells in CD40L-containing downstream differentiation media formulations exhibited a marked increase in the output of CD19- and IgM-expressing cells (in terms of yield) in comparison to a control condition.

### Example 6: Maintenance of PSC

Where hPSCs were used to derive B cell precursors, they were maintained on Matrigel™ coated plates in either mTeSR™1 (STEMCELL Technologies), TeSR™-E8 (STEMCELL Technologies), or mTeSR™ Plus (STEMCELL Technologies) media for 6-8 days, in accordance with the manufacturer's recommendations. Complete media changes were performed as needed. PSC colonies were clump passaged onto freshly coated Matrigel™ plates in maintenance culture. Where the PSC were used for downstream assays, the colonies were dissociated using ACCUTASE™ (STEMCELL Technologies) to obtain a single cell suspension, in accordance with manufacturer's recommended protocol.

### Example 7: Formation of PSC Aggregates and Differentiation

Prior to obtaining the single cell suspension in accordance with Example 6, 24-well or 6-well Aggrewell™ 400 plates (STEMCELL Technologies) were prepared in accordance with the manufacturer's recommendations, including reducing adherence of cells for the microwell devices using the anti-adherence rinsing solution (STEMCELL Technologies). Following the recommended incubation, the anti-adherence rinse solution was discarded and each well was rinsed once with an equal volume of DMEM-F12 with 15 mM HEPES.

After preparing the microwell device, the hPSCs dissociated in accordance with Example 6 were seeded into one or more wells of the microwell device in EB Formation Medium (STEMdiff™ Hematopoietic-EB Basal Medium supplemented with STEMdiff™ Hematopoietic-EB Supplement A (STEMCELL Technologies)) and 10 μM Y-27632 (STEMCELL Technologies). In embodiments using the 6-well format of the microwell device, 2.5 mL of EB Formation Medium was added to a well of the microwell device. Next, a further 2.5 mL of a cell suspension (^{˜}1.4×10⁶ cells/mL) in EB Formation Medium was added to the well and the microwell device was briefly centrifuged and incubated at 37° C. If using a 24-well format of the microwell device, then the volume per well should be scaled down accordingly to 2 mL/well. The final cell concentration in each well of the microwell device should be ^{˜}3×10⁵ cells/ml, or 6×10⁵ cells/well of a 24-well plate or 7×10⁵ cells/mL, or 3.5×10⁶ cells/well of a 6-well plate.

### Example 8: Differentiating Aggregates to Hematopoietic Progenitors

Aggregates were prepared as described in Example 7, and on day 2 after forming the aggregates 2.5 mL of medium in each well of the microwell device was carefully removed and discarded without disturbing the aggregates. A 2.5 mL volume of fresh EB Medium A (STEMdiff™ Hematopoietic-EB Basal Medium (STEMCELL Technologies) supplemented with STEMdiff™ Hematopoietic-EB Supplement A (STEMCELL Technologies)) was added to each well and the microwell device was incubated at 37° C.

On day 3 when mesoderm intermediates (e.g. mesoderm precursors) are formed, 2.5 mL of medium in each well of the microwell device was carefully removed and discarded without disturbing the aggregates. A 2.5 mL volume of fresh EB Medium B (STEMdiff™ Hematopoietic-EB Basal Medium (STEMCELL Technologies)) supplemented with STEMdiff™ Hematopoietic-EB Supplement B (STEMCELL Technologies) was added to each well and incubated at 37° C. to differentiate the mesodermal precursor cells to hematopoietic progenitors.

On day 5, the aggregates were harvested from each well of the microwell device and passed through a 37 μm reversible filter (STEMCELL Technologies) to isolate the aggregates on a surface thereof. The filtrate of aggregates was deposited into a fresh tube by inverting the filter over the fresh tube and directing 2.5 mL/well fresh EB Medium B against the mesh (1 mL/well of a 24-well format of the microwell device was used). Thusly obtained aggregates were gently resuspended before adding the full volume to a non-tissue culture-treated plate and then incubated at 37° C. Each well of the 6-well plate was topped up with 2.5 mL fresh EB Medium B (or 1 mL/well of a 24-well plate) on day 7 and then incubated at 37° C. On day 10, a half-medium change with fresh EB Medium B was performed taking care not to disrupt the aggregates and then incubated at 37° C. for a further 2 days.

### Example 9: Enriching Hematopoietic Progenitors

The aggregates of Example 8 were harvested from each well and transferred to individual 15 mL tubes. The tubes were centrifuged at 300×g for 5-10 minutes. The supernatant was aspirated and 1 mL of Collagenase Type II—2500 U/mL (STEMCELL Technologies) (Cat #07418) was added to each tube and incubated at 37° C. for 20 minutes. Following, 3 mL of TryPLE™ Express was added and each tube was incubated for an additional 20 minutes.

After the incubation, each tube was topped-up with 6 mL of DMEM/F12 and filtered through a 37 μm filter. The eluate was centrifuged at 300×g for 5-10 minutes and the supernatant was discarded. The pelleted cells were subjected to a CD34⁺ enrichment protocol (EasySep™ Human CD34 Positive Selection Kit II, STEMCELL Technologies). The manufacturer's recommendations for the CD34⁺ enrichment were followed except the number of magnetic separations was reduced from 4 to 2. H1, H9, WLS-1C, STiPS-M001, and STiPS-F016 PSC lines efficiently differentiated to CD34⁺ hematopoietic progenitor cells.

### Example 10: Deriving B Cell Precursors from hPSC-Derived CD34⁺ HSPC

The enriched CD34⁺ HSPC of Example 9 were seeded at a density of 2.5×10⁴ cells/well of a 24-well plate, and cultured for 14 days in a derivation medium and in the presence of various coatings of extracellular matrix proteins or cell adhesion molecules, or MS-5 stromal cells. Derivation medium typically comprised a basal medium, such as StemSpan™ SFEM II (STEMCELL Technologies), and an assortment of stage-specific cytokines and growth factors, as in Examples 3 and 4. An initial iteration of a derivation medium was formulated as described above with the further inclusion of IGF-1, and after 14 days the output H9-derived cells were analyzed for CD10 and CD19 expression.

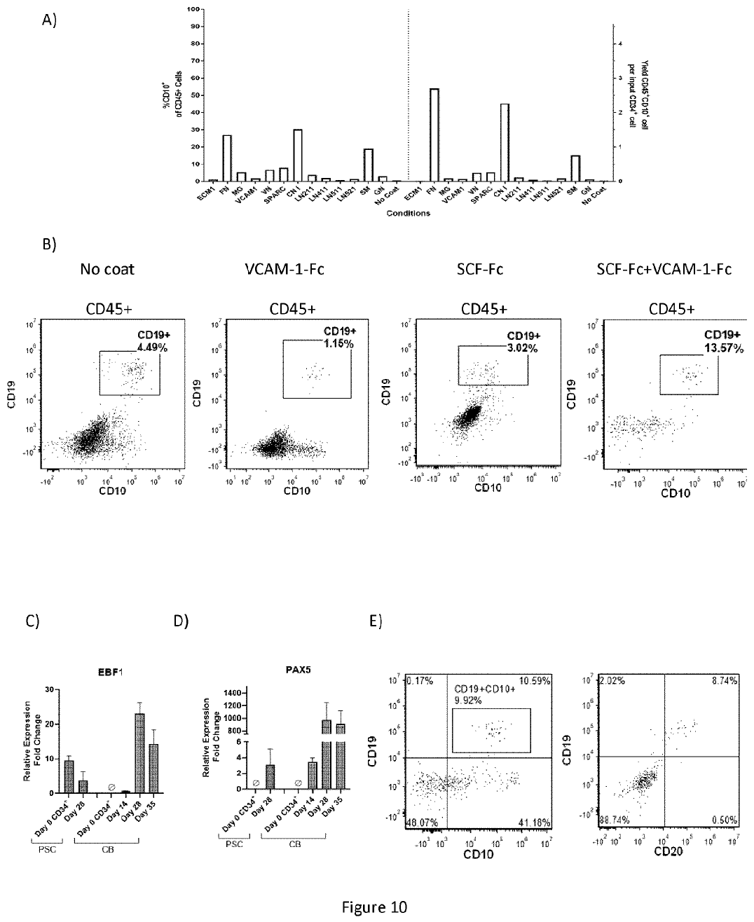

The highest frequency of B cell precursors was generated when the hPSC were cultured in wells coated with either fibronectin or collagen1, outperforming more traditional approaches such as Matrigel™ coating (FIG. 10A) or on MS-5 stromal cells (data not shown). It was also observed that an appreciable frequency of B cell precursors was generated when the hPSC were cultured in wells coated with no extracellular matrix or coated with one or both of VCAM-1 and SCF-Fc (FIG. 10B).

The PSC derived CD34⁺ HSPC cells were investigated for their capability to differentiate into B lineage cells by comparing expression of marker transcription factors in such cells to the expression levels in cord blood derived CD34⁺ HSPC cells. It was observed that the relative fold change expression of the B cell specification transcription factor EBF1 decreased over time in both the PSC derived CD34⁺ HSPC and the cord blood derived CD34⁺ HSPC cells (FIG. 10C). Fold change expression of the B cell commitment factor PAX5 increased during the differentiation protocol in both the PSC derived CD34⁺ HSPC and the cord blood derived CD34⁺ HSPC cells (FIG. 10D), suggesting that the PSC-derived differentiated cells had a similar transcriptional program to cord blood-derived counterparts.

### Example 11: Differentiating CD19⁺ B Lineage Cells from hPSC-Derived B Cell Precursors

B cell precursors generated as described in Example 10 on VCAM-1 and/or SCF-Fc coated or uncoated plates were re-seeded at 5.0×10⁴ cells/well of a 24-well plate on VCAM-1 coated plates and cultured for 14 days in differentiation medium, essentially as described in Example 4 but with the further inclusion of IGF-1. Day 28 H9-derived cells were harvested and analyzed by flow cytometry for expression of CD10 and CD19 (FIG. 10E) and for expression of CD19 and CD20 (FIG. 10E).

The population of CD19 expressing H9-derived cells also expressed CD20, suggesting that the described serum- and feeder-cell free conditions could generate CD19⁺ B lineage cells.

### Example 12: Generating B Cell Precursors and CD19⁺ B Lineage Cells from Cord Blood-Derived CD34⁺ HSPC in the Presence of a Coating

The effect of extracellular matrix protein coating or cell adhesion molecule coating (in comparison to no coating) was also tested in the context of deriving B cell precursors and differentiating the B cell precursors to CD19⁺ B lineage cells from pop1 cells.

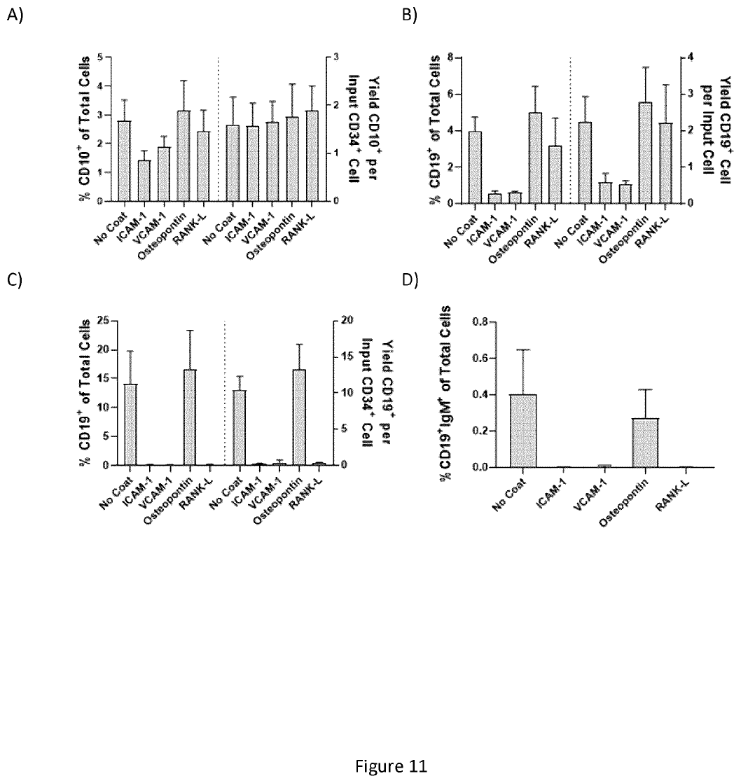

Briefly, the pop1 cells were sorted as described in Example 1 and seeded at a density of 5000 cells per well of a 24-well plate into a TPO-free derivation medium, essentially as described in Example 3 (FIG. 2). After 14 days of culture in the derivation medium in the presence of the different coatings, each output population of cells was harvested and analyzed by flow cytometry for CD10⁺ (FIG. 11A) and CD19⁺ (FIG. 11B) frequency and yield. Next, the day 14 cells were re-seeded at 1-1.5×10⁵ cells per well in correspondingly coated wells of a 24-well plate, and cultured in the presence of a differentiation medium, essentially as described in Example 4. Day 28 cells were harvested and analyzed by flow cytometry for CD19⁺ cell frequency and yield (FIG. 11C) and IgM⁺ cell frequency among the CD19⁺ cells (FIG. 11D).

The tested coatings did not appear to have a significant impact on derivation of day 14 B cell precursors, and did not positively impact the generation of CD19 expressing cells on day 14. Interestingly, it appeared that certain of the tested coatings may be detrimental to the generation of CD19-expressing cells on day 14. The same general conclusions could be drawn in regard to the generation of CD19⁺ B lineage cells on day 28 and generation of IgM-expressing cells on day 28.

1. A method for differentiating a population of B cell precursors, comprising:
contacting a population of CD34⁺ hematopoietic stem or progenitor cells (HSPC) with a derivation medium comprising a basal medium, at least one of stem cell factor (SCF), thrombopoietin (TPO), and FMS-like tyrosine kinase 3 ligand (FLT3L), and at least one other cytokine; and
culturing the population of HSPC in the derivation medium under serum-free conditions to obtain a population of B cell precursors,
wherein the population of B cell precursors express one or both of CD10 or CD19.

2. The method of claim 1, wherein the population of HSPC are enriched from a tissue source or are differentiated from pluripotent stem cells (PSC).

3. (canceled)

4. The method of claim 1, wherein the derivation medium comprises either SCF or TPO.

5. The method according to claim 1,
further comprising contacting the population of B cell precursors with a differentiation medium comprising a basal medium, at least one of SCF, TPO, and FLT3L, and at least one other cytokine; and
culturing the population of B cell precursors in the differentiation medium under serum-free conditions.

6. The method according to claim 5, further comprising obtaining a population of CD19⁺ B lineage cells having more CD19⁺ cells than after culturing the population of HSPC in the derivation medium.

7. (canceled)

8. The method according to claim 5, wherein at least a fraction of the CD19⁺ B lineage cells are IgM⁺ cells.

9. (canceled)

10. The method according to claim 6, further comprising:
contacting the population of CD19⁺ B lineage cells with a downstream differentiation medium comprising a basal medium, a ligand of human CD40, and the at least one other cytokine; and
culturing the population of CD19⁺ B lineage cells in the downstream differentiation medium under serum-free conditions.

11. The method according to claim 10, further comprising obtaining more IgM⁺ cells than after culturing the population of B cell precursors in the differentiation medium.

12. The method according to claim 11, wherein at least a fraction of the IgM⁺ cells are antibody secreting cells.

13. (canceled)

14. A method for differentiating a population of B lineage cells, comprising:
contacting a population of B cell precursors with a differentiation medium comprising a basal medium, at least one of SCF, TPO and FLT3L, and at least one other cytokine; and
culturing the population of B cell precursors in the differentiation medium under serum-free conditions to obtain a population of B lineage cells,
wherein the population of B cell precursors express one or both of CD10 or CD19.

15. (canceled)

16. The method of claim 14, wherein the population of B cell precursors are derived from a population of CD34⁺ hematopoietic stem or progenitor cells (HSPC) that are either enriched from a tissue source or are differentiated from pluripotent stem cells (PSC).

17. The method according to claim 16, wherein the population of B lineage cells express CD19 and the population of B lineage cells comprises more CD19⁺ cells than after culturing the population of HSPC in a serum-free derivation medium comprising a basal medium, at least one cytokine, and one or more of SCF, TPO, and FLT3L.

18. The method according to claim 17, wherein at least a fraction of CD19⁺ B lineage cells are IgM⁺ cells.

19. The method according to claim 14, further comprising:
contacting the population of B lineage cells with a downstream differentiation medium comprising a basal medium, a ligand of human CD40, and the at least one other cytokine; and
culturing the population of B lineage cells in the downstream differentiation medium under serum-free conditions.

20. The method according to claim 19, further comprising obtaining more IgM⁺ cells than after culturing the population of B cell precursors in the differentiation medium.

21. The method according to claim 20, wherein at least a fraction of the IgM⁺ cells are antibody secreting cells.

22-24. (canceled)

25. The method of claim 1, wherein the at least one other cytokine is
i) one or more of IL-3, IL-6, or IL-7, or
ii) one or more of IL-2, IL-3, IL-4, IL-6, IL-7, IL-10 or IL-21.

26. The method of claim 14, wherein the at least one other cytokine is
i) one or more of IL-3, IL-6, or IL-7, or
ii) one or more of IL-2, IL-3, IL-4, IL-6, IL-7, IL-10, or IL-21.

27. The method according to claim 1, wherein the methods are performed under feeder cell-free conditions.

28-42. (canceled)

43. The method according to claim 14, wherein the methods are performed under feeder cell-free conditions.
